(19) United States
(12) Patent Application Publication
(10) Pub. No.: US 2009/0305259 A1
(43) Pub. Date: Dec. 10, 2009

(54) Early Diagnosis of Congenital Abnormalities in the Offspring of Diabetic Mothers

(75) Inventors: Joseph Madri, North Branford, CT (US); Anjali Nath, Guilford, CT (US); Michael Krauthammer, New Haven, CT (US); Michael Snyder, Fairfield, CT (US); Eugene Davidov, Hamden, CT (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: YALE UNIVERSITY, New Haven, CT (US)

(21) Appl. No.: 12/281,767

(22) PCT Filed: Mar. 21, 2007
(86) PCT No.: PCT/US2007/006966

§ 371 Date: Jul. 21, 2009

Related U.S. Application Data
(60) Provisional application No. 60/784,847, filed on Mar. 21, 2006.

(51) Int. Cl. C12Q 1/68; G01N 33/53; G01N 33/00; C12Q 1/54; C12Q 1/37; C12Q 1/02

(52) U.S. Cl. 435/6; 435/7.1; 435/7.92; 435/14; 435/23; 435/29

(57) ABSTRACT

The present invention relates to the identification of a series of biomarkers, the detection of which is prognostic for women at risk of becoming hyperglycemic during pregnancy and/or fetuses at risk of developing congenital anomalies as a result of maternal hyperglycemia.

### BACKGROUND OF THE INVENTION

Diabetes affects approximately 8% (320,000) of human pregnancies annually in the US. Hyperglycemia in pregnant women is associated with a decreased pregnancy rate, increased post-implantation loss and a 2- to 5-fold increase in congenital abnormalities that have a 20-50% mortality rate compared to the general population.

Diabetic embryopathy (birth defects and spontaneous abortions) results from maternal metabolic abnormalities during the first 6-7 weeks of gestation. The embryopathy appears to be multifactorial in origin, and the resulting defects remain important causes of morbidity and mortality in diabetic pregnancies.

Clinical data suggests that the teratogenic effects of hyperglycemia may be blunted by meticulous control of maternal glucose levels. However, patients with maintained euglycemia throughout the period of organogenesis still do not achieve mortality and morbidity rates comparable to the non-diabetic population Data suggests that even transient bouts of hyperglycemia, even in normal pregnancies, may be sufficient to cause pregnancy loss or congenital defects if it occurs during critical periods early in pregnancy.

While no diabetes-specific constellation of defects has emerged, the anomalies most frequently encountered affect the central nervous system, heart and great vessels, kidneys and axial skeleton. In the Baltimore-Washington Infant study (BWIS), a population-based case-control study of cardiovascular malformations (CVM), an early analysis of all types of CVM revealed a three-fold increased risk in infants of mothers with pre-gestational diabetes (Ferenze, Epidemiology of congenital heart disease: the Baltimore-Washington Infant Study, 1981-1989. Mt. Kisko, N.Y.: Futura Publishing). The risk was greatest for specific phenotypes representing defects of primary cardiogenesis, i.e. transposition of the great vessels, ventricular septal defect, atrial septal defect, tetralogy of Fallot, coarctation of the aorta, single umbilical artery, hypoplastic left heart, and cardiomegaly.

The mechanisms of hyperglycemic teratogenesis have not been fully elucidated, but a range of biological processes have been identified as being dysregulated in the offspring of diabetic mothers as well as conceptus cultures of rats and mice exposed to elevated glucose concentrations comparable to those observed in diabetic mothers (Lee, 1995, Diabetes, 44:20-24). Increased oxidative stress in the embryos leads to reduced expression of several genes, e.g. Pax3, a gene involved in neural tube fusion and p53-mediated apoptosis (Loeken, 2006, J. Soc. Gynecol. Investig., 13:2-10). In addition, hyperglycemia evokes changes in sorbitol, reactive oxygen species (ROS), arachidonic acid, collagen expression, MMP-2, superoxide dismutase, inducible nitric oxide synthase (iNOS) and epithelial nitric oxide synthase (eNOS). Vascular endothelial growth factor (VEGF) is also dysregulated in glucose treated embryos and the embryos of streptozotocin induced diabetic pregnant mice. Hyperglycemia is also associated with changes in protein phosphorylation including that of adhesion molecules such as platelet derived endothelial cell adhesion molecule (PECAM-1).

Cardiovascular defects that arise as a result of a disruption in normal embryological patterning are among some of the most devastating defects that result from hyperglycemia during pregnancy. Identifying molecular targets of hyperglycemia induced dysregulation would significantly enhance our understanding of the normal developmental program that governs cardiovascular organogenesis and also potentially identify therapeutic candidates.

Increased α-D-glucose in diabetic rodent models interrupts the epithelial-mesenchymal transformation necessary for normal formation of the endocardial cushion. The endocardial cushion is a precursor of the atrioventricular valves and a portion of the atrioventricular speta. Formation of the endocardial cushion occurs via an epithelial-mesenchymal transformation in which a subpopulation of endothelial cells within the endocardial layer adjacent to the atrioventricular canal down regulate cell adhesion molecules (Mjaatvedt and Markwald, 1989, Dev. Biol., 136:118-128), separate from the endocardium and transform into migratory mesenchymal cells that invade the underlying cardiac jelly (Runyan and Markwald, 1983, Dev. Biol., 95:108-114); these cardiovascular abnormalities can give rise to lethal phenotypes because of abnormal vascular patterning (Madri, 2003, Pediatric Dev. Pathol., 6:334-341.)

Establishment of the extra-embryonic vascular system is an essential developmental step, vital for survival, growth and homeostasis of vertebrate embryos. Vasculogenesis, the in situ development of blood vessels from angioblasts, begins in the extra-embryonic yolk sac prior to vasculogenesis in the embryo proper. The yolk sac serves an essential function at the maternal-fetal interface and is vital for the normal progression of cardiovascular development. Development of vitelline circulation allows the embryo to shift from its reliance on diffusion-dependent nutrient delivery to a system of vascular conduits (Jollie, 1990, Teratology, 41:361-381). Studies in knock-out mice have revealed a number of essential molecules that participate in vasculogenesis including ephrins, TIE2, angiopoetin, PECAM, VEGF and basic fibroblast growth factor (bFGF) (Gerety and Anderson, 2002, Development, 129:1397-1410; Suri, 1996, Cell, 87:1171-1180; Gerber, 1999, Mech. DeV., 80:77-86; Yasuda, 1992, Dev. Biol., 150:397-413). These knock out animals are characterized by enlarged, hyperfused capillaries and embryonic lethality due to cardiovascular defects.

In rat and mouse models, increased α-D-glucose in pregnant diabetic females arrests yolk sac vasculogenesis, correlated with reduced VEGF-A mRNA and protein levels (Pinter, 2001, Am. J. Pathol., 158:1199-1206). VEGF-A is critical to normal cardiovascular development; modest changes in VEGF-A levels in the yolk sac and heart lead to embryonic vasculopathy (Carmeliet, 1996, Nature, 380:435-439; Miquerol, 2000, Development, 127:3941-3946; Damert, 2002, Development, 129:1881-1892). Hyperglycemia leads to reduced VEGF-A expression (Pinter, 2001, Am. J. Pathol., 158:1199-1206). VEGF-A in turn modulates PECAM-1 expression and its phosphorylation state; persistent PECAM-1 expression., a failure to up-regulate MMP-2 expression and consequently vascular abnormalities are all sequelae of hyperglycemia.

Nitric oxide (NO) is generated by three conserved nitric oxide synthases (NOS): isozymes: endothelial NOS (eNOS), inducible NOS (iNOS) and neuronal NOS (nNOS). The control of NOS isoform expression is complex involving transcriptional and post-transcriptional mechanisms triggered by multiple signaling pathways during development, growth and homeostasis as well as pathophysiological conditions. NOS isoform subcellular localization, association with receptors and catalysis are tightly regulated by a diverse number of binding proteins with various specificities (e.g. calmodulin, dynamin-2, caveolin-1).

NOSs catalyze the oxidation of L-arginine to NO and L-citrulline, using the cofactors NADPH, FAD and BH₄. NO can freely diffuse through membranes and enter the extracellular space, as far as 100 μm in a few seconds at 37° C. Diffusion of NO is limited only by its chemical half life and reactivity. NO modifies thiols via S-nitrosolyation or disulfide formation, or cysteine residues and can also interact with free radicals to induce a variety of biological responses. NO is a pleiotropic molecule that plays a variety of roles in numerous systems especially via the activation of guanylate cyclase as well as the activation of several other kinases. NO has also been found to play a role in the development and differentiation of several organ systems.

An inverse relationship between eNOS and iNOS isoforms exists during vasculargenesis in the yolk sac in mice. During the earliest stage (blood island formation occurring from embryonic day (E)7-8), iNOS protein levels are high while eNOS is barely detectable. At E8-8.5, (the primary capillary plexus stage of vasculogenesis) a switch in the expression levels of e-NOS and iNOS occurs. At E8.5-9.5 vessel maturation and remodeling occurs and iNOS protein is absent while eNOS expression is maintained. Hyperglycemia in the form of 20 mM D-glucose to in vitro cultures of rat and mouse conceptuses at the primitive streak phase of development results in significant yolk sac and embryonic vasculopathy. This vasculopathy is correlated with iNOS up-regulation and its inappropriate maintenance throughout vasculogenesis, while eNOS expression levels are decreased relative to controls.

Administration of an exogenous NO donor to these cultures restores the normal relative expression patterns of iNOS and eNOS. In addition, administering NO donors to these cultures rescued the glucose induced vitelline vasculo- and angiogenesis process (Nath, 2004, Development, 131:2485-2496), suggesting a potential therapeutic use for NO donors in the treatment of maternal diabetes-induced embryonic vasculopathy.

The complexity of the regulatory mechanisms which govern development of the heart and associated vessels in the embryo is not fully understood. Teasing out each molecule involved in the process and understanding its role in the developmental program has been laborious and in many cases, inconclusive. The present invention fills a long outstanding need in the art for identifying candidate biomarkers that are dysregulated by hyperglycemia during pregnancy and are prognostic for clinical outcomes for both mother and fetus.

### SUMMARY OF THE INVENTION

The present invention provides compositions and methods of identifying a pregnant female who is at risk of experiencing hyperglycemia, who is at risk of developing gestational diabetes, and/or whose fetus is at risk of developing a congenital abnormality.

In one embodiment of the invention, a method of identifying a pregnant female whose fetus is at risk of developing a congenital abnormality comprises measuring the level of at least one biomarker in a body sample obtained from a female. When the level of the biomarker measured in the sample indicates that the biomarker is dysregulated in the female, the pregnant female's fetus is at risk for developing a congenital abnormality.

In one aspect of the invention, the pregnant female is a mammal selected from the group consisting of a mouse, a rat, a non-human primate, and a human. In a preferred aspect of the invention, the mammal is a human.

In one embodiment of the invention, the method comprises measuring the level of two or more biomarkers in a body sample, wherein the biomarker is selected from the group consisting of a matrix metalloproteinase, a receptor, a ligand, a transcription factor, a protein affecting apoptosis, a cytoskeletal protein, a cell adhesion molecule, actin, a mictotubule protein, an enzyme, a metabolite associated with glucose metabolism, and a metabolite associated with diabetes.

In a preferred embodiment of the invention, the biomarker is selected from the group consisting of laminin γ1 chain, laminin α4 chain, ADAM 15; MMP-2, MMP-9, Wnt16, enolase 1α, Down syndrome critical region protein, ST14, CH3T, SVCT, NG2, NOGO A, and PC1/3. In one aspect of the invention, the body sample is selected from the group consisting of a tissue, a cell and a bodily fluid. In another aspect of the invention, the bodily fluid comprises maternal serum or amniotic fluid.

In one embodiment of the invention, measuring of the biomarker comprises an immunoassay for assessing the level of the biomarker in a body sample, wherein the immunoassay is selected from the group consisting of Western blot, ELISA, immunopercipitation, immunohistochemistry, immunofluorescence, radioimmunoassay, dot blotting, and FACS.

In another embodiment of the invention, measuring of the biomarker comprises a nucleic acid assay for assessing the level of a nucleic acid encoding the biomarker in a body sample, wherein the nucleic assay is selected from the group consisting of a Northern blot, Southern blot, in situ hybridization, a PCR assay, an RT-PCR assay, a probe array, a gene chip, and a microarray.

Another embodiment of the invention includes a method of identifying a pregnant female whose is at risk of developing hyperglycemia during pregnancy. The method comprises measuring the level of at least one biomarker in a body sample obtained from a pregnant female, wherein when the level of the biomarker in the sample indicates that the biomarker is dysregulated, the pregnant female is at risk for developing hyperglycemia. In one aspect of the invention, the pregnant female is a mammal selected from the group consisting of a mouse, a rat, a non-human primate, and a human. In a more preferred aspect of the invention, the mammal is a human.

In one aspect of the invention, the method of the invention comprises measuring the level of two or more biomarkers in said body sample, wherein said biomarker is selected from the group consisting of a matrix metalloproteinase, a receptor, a ligand, a transcription factor, a protein affecting apoptosis, a cytoskeletal protein, a cell adhesion molecule, actin, a mictotubule protein, an enzyme, a metabolite associated with glucose metabolism, and a metabolite associated with diabetes. In a preferred aspect of the invention, the biomarker is selected from the group consisting of laminin γ1 chain, laminin α4 chain, ADAM 15; MMP-2, MMP-9, Wnt16, enolase 1α, Down syndrome critical region protein, ST14, CH3T, SVCT, NG2, NOGO A, and PC1/3. In one aspect of the invention, the body sample is selected from the group consisting of a tissue, a cell and a bodily fluid. In another aspect of the invention, the bodily fluid comprises maternal serum or amniotic fluid.

In one embodiment of the invention, measuring of the biomarker comprises an immunoassay for assessing the level of the biomarker in the sample. In another embodiment of the invention, measuring of the biomarker comprises a nucleic acid assay for assessing the level of a nucleic acid encoding the biomarker in the sample.

Another embodiment of the invention provides a method of identifying an individual whose is at risk of developing hyperglycemia, the method comprising measuring in a body sample obtained from the individual the level of at least one biomarker, wherein when the level of the biomarker in the sample indicates that the biomarker is dysregutated in said individual, said individual is at risk for developing hyperglycemia. In one aspect of the invention, the individual is a mammal selected from the group consisting of a mouse, a rat, a non-human primate, and a human. In a preferred aspect of the invention, the mammal is a human.

In yet another embodiment of the invention, the method comprises measuring the level of two or more biomarkers in said body sample, wherein said biomarkers are selected from the group consisting of a matrix metalloproteinase, a receptor, a ligand, a transcription factor, a protein affecting apoptosis, a cytoskeletal protein, a cell adhesion molecule, actin, a mictotubule protein, an enzyme, a metabolite associated with glucose metabolism, and a metabolite associated with diabetes. In a preferred embodiment of the invention, the biomarker is selected from the group consisting of laminin γ1 chain, laminin α4 chain, ADAM 15; MMP-2, MMP-9, Wnt16, enolase 1α, Down syndrome critical region protein, ST14, CH3T, SVCT, NG2, NOGO A, and PC1/3. In one aspect of the invention, the body sample is selected from the group consisting of a tissue, a cell, and a bodily fluid.

In one embodiment of the invention, measuring the biomarker comprises an immunoassay for assessing the level of the biomarker in the sample. In another embodiment of the invention, measuring the biomarker comprises a nucleic acid assay for assessing the level of a nucleic acid encoding the biomarker in the sample.

In yet another embodiment, the invention includes a composition comprising a plurality of oligonucleotides attached to a substrate surface, wherein each of the oligonucleotides is a nucleic acid encoding a biomarker or a fragment thereof, or is complementary to the biomarker or the fragment thereof, wherein the biomarker is selected from the group consisting of a matrix metalloproteinase, a receptor, a ligand, a transcription factor, a protein affecting apoptosis, a cytoskeletal protein, a cell adhesion molecule, actin, and a mictotubule protein, an enzyme, a metabolite associated with glucose metabolism, and a metabolite associated with diabetes. In still another embodiment, the invention includes the biomarker attached to a substrate surface, wherein the substrate surface is a membrane, a chip, a bead, a microsphere or a microchip. In a preferred embodiment, the composition comprises a biomarker selected from the group consisting of laminin γ1 chain, laminin α4 chain, ADAM 15; MMP-2, MMP-9, Wnt16, enolase 1α, Down syndrome critical region protein, ST14, CH3T, SVCT, NG2, NOGO A, and PC1/3.

In another embodiment, the invention includes a composition comprising a plurality of peptides attached to a substrate surface, wherein each of the peptides is a biomarker or a fragment thereof, wherein the biomarker is selected from the group consisting of a matrix metalloproteinase, a receptor, a ligand, a transcription factor, a protein affecting apoptosis, a cytoskeletal protein, a cell adhesion molecule, actin, a mictotubule protein, an enzyme, a metabolite associated with glucose metabolism, and a metabolite associated with diabetes. In one aspect, the biomarker is attached to a substrate surface, wherein the substrate surface is a membrane, a chip, a bead, a microsphere or a microchip. In another aspect of the invention, each of the peptides is a biomarker or a fragment thereof, wherein the biomarker is selected from the group consisting of laminin γ1 chain, laminin α4 chain, ADAM 15; MMP-2, MMP-9, Wnt16, enolase 1α, Down syndrome critical region protein, ST14, CH3T, SVCT, NG2, NOGO A, and PC1/3.

In yet another embodiment, the invention includes a composition comprising a plurality of antibodies attached to a substrate surface wherein the antibody specifically binds a biomarker or a fragment thereof, wherein the biomarker is selected from the group consisting of a matrix metalloproteinase, a receptor, a ligand, a transcription factor, a protein affecting apoptosis, a cytoskeletal protein, a cell adhesion molecule, actin, a mictotubule protein, an enzyme, a metabolite associated with glucose metabolism, and a metabolite associated with diabetes. In one aspect, the composition comprises a substrate surface, wherein the substrate surface is a plate, a membrane, a solid support, a chip, a bead, a microsphere or a microchip. In another aspect of the invention, the composition comprises an antibody that specifically binds a biomarker or a fragment thereof, wherein the biomarker is selected from the group consisting of laminin γ1 chain, laminin α4 chain, ADAM 15; MMP-2, MMP-9, Wnt16, enolase 1α, Down syndrome critical region protein, ST14, CH3T, SVCT, NG2, NOGO A, and PC1/3.

In still another aspect of the invention, at least one of the antibodies is attached to a substrate surface. In a preferred aspect of the invention, two or more antibodies are attached to a substrate surface. More preferred, the antibody comprises a detectable label, wherein the detectable label is selected from the group consisting of a radioactive, a fluorescent, a biological, and an enzymatic label.

Still another embodiment of the invention includes a kit comprising a composition for detecting the level of a biomarker in a body sample obtained from a mammal, wherein when the level of the biomarker in the sample indicates that the biomarker is dysregulated in the individual, the individual is at risk of developing hyperglycemia, and wherein the composition comprises at least one antibody that specifically binds the biomarker or a fragment thereof, the kit further comprising instructional material for the use thereof. In one aspect of the invention, the mammal is a human. In another aspect of the invention, the human is a female. In still another aspect, the female is pregnant.

In one aspect of the invention, the kit composition comprises at least one antibody that specifically binds a biomarker or a fragment thereof, wherein the biomarker is selected from the group consisting of laminin γ1 chain, laminin α4 chain, ADAM 15; MMP-2, MMP-9, Wnt16, enolase 1α, Down syndrome critical region protein, ST14, CH3T, SVCT, NG2, NOGO A, and PC1/3. In another aspect of the invention, at least one of the antibodies is bound to a substrate surface. In still another aspect of the invention, two or more of the antibodies are bound to the substrate surface. In yet another aspect of the invention, the antibody comprises a detectable label, wherein the detectable label is selected from the group consisting of a radioactive, a fluorescent, a biological, and an enzymatic label.

Another embodiment of the invention includes a kit comprising a composition for detecting the level of a biomarker in a body sample obtained from a mammal, wherein when the level of the biomarker in the sample indicates that the biomarker is dysregulated in the individual, the individual is at risk of developing hyperglycemia, and wherein the composition comprises at least one nucleic acid, wherein the nucleic acid encodes the biomarker or a fragment thereof, or is complementary to the biomarker or a fragment thereof, the kit further comprising an instructional material for the use thereof. In one aspect of the invention, the mammal is a human. In another aspect of the invention, the human is a female. In still another aspect of the invention, the female is pregnant. In a preferred embodiment, the biomarker is selected from the group consisting of laminin γ1 chain, laminin α4 chain, ADAM 15; MMP-2, MMP-9, Wnt16, enolase 1α, Down syndrome critical region protein, ST14, CH3T, SVCT, NG2, NOGO A, and PC1/3. In one aspect of the invention, the nucleic acid probe is immobilized on a solid support. In another aspect of the invention, the nucleic acid probe is linked to a detectable label, wherein the label is selected from a radioactive, a fluorescent, a biological and an enzymatic label.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A through 1E, is a series of images demonstrating that high alpha-D-glucose levels (20 mM) arrest yolk sac vasculogenesis at the primary capillary plexus stage. FIGS. 1A and 1B are low power representative micrographs of PECAM-1 stained 9.5 days post coitus (dpc) yolk sacs harvested from normoglycemic (A) and hyperglycemic (B) cultures. FIG. 1C is a bar graph depicting altered expression of VEGF-A₁₆₅ which correlates with the lack of vascular arborizations seen in FIG. 1B. FIGS. 1D and 1E are bar graphs illustrating the rescue of this arrested phenotype (as evidenced by arborization of the yolk sac vasculature similar to panel (A) and functional circulation observed in real time by treatment with exogenous VEGF-A₁₆₅ at a specific concentration range (D & E). FIG. 1D illustrates an effect of exogenous VEGF-A₁₆₅ at a concentration above 2 pg on vascular development. FIG. 1E illustrates an effect of VEGF-A₁₆₅ on the arrest of vascular development induced by hyperglycemia.

FIG. 2, comprising FIGS. 2A through 2E, is a series of images demonstrating the effects of hyperglycemia on iNOS/eNOS distribution and NO production, and rescue by NO donor. FIG. 2A is a photomicrograph illustrating PECAM-1 staining at 9.5 dpc in cultures exposed to hyperglycemia. FIG. 2B is a photomicrograph that illustrates the ability of NOC-18 (a slow-release NO Donor) to restore normal vascular morphology of hyperglycemic treated conceptuses by PECAM-1 staining at 9.5 dpc. FIG. 2C is a graph depicting Erk-2 normalized iNOS expression in pooled 7.5, 8.5 and 9.5 dpc conceptuses. FIG. 2D is a graph of Erk-2 normalized eNOS protein expression in pooled 7.5, 8.5 and 9.5 dpc conceptuses. The dashed lines represent the hyperglycemic condition while the solid black lines represent the control (n=4, *p<0.05, **p<0.01). The data (mean±S.E.M) is relative to the control 7.5 dpc levels. Above each graph is a representative Western blot of iNOS and eNOS at 8.5 dpc. FIG. 2E is a representative Western blot of the effect of a NO donor on NOS protein distribution in glucose treated conceptuses at 8.5 dpc and a graph of Erk-2 normalized averaged data for eNOS and iNOS protein expression (open columns represent eNOS and black columns represent iNOS; averages of two experiments). Normoglycemic=Nml, hyperglycemic=HG, and hyperglycemic+NOC-18=HG+NOD (Nath, 2004, Development, 131:2485-2496).

FIG. 3, comprising FIGS. 3A through 3D, is a series of images of in vitro culture of murine concepti illustrating the development of the vitelline vasculature and organogenesis. The Yolk Sac is observed to consist of an endodermal layer (En) overlying a layer of mesoderm (Mes), from which will differentiate angioblasts, which will further differentiate into the endothelia of the Yolk Sac vasculature (Vasc). FIGS. 3A and 3B are photomicrographs of concepti at 7.5 dpc. FIGS. 3C and 3D are photomicrographs of concepti at 9.5 dpc.

FIG. 4, comprising FIG. 4A and FIG. 4B, is a series of images illustrating yolk sac vascular development in normal- or hyperglycemic conditions. FIG. 4A is a photomicrograph depicting representative “en face” PECAM-1 labeled yolk sac depicting vascular development at 8.5 dpc. FIG. 4B is a representative silver stained SDS-PAGE of lysates derived from normoglycemic (control), hyperglycemic (20 mM alpha-D-glucose) (HG), HG+VEGF A₁₆₅ and HG+Noc-18 treated cultures; (n=10 for each group).

FIG. 5, is a series of charts depicting a series of representative total ion chromatograms. Peptide mass spectra are derived from yolk sac extracts in normoglycemic (NG) or hyperglycemic (HG) conditions.

FIG. 6 is a representative window of the MZmine proteomic analysis software that is used for data preprocessing to align and compare peak intensities across all samples simultaneously. NG=euglycemic; HG=hyperglycemic; HG+VEGF=hyperglycemic+rVEGF; HG+NOD=hyperglycemic+NO donor. YS#=yolk sac extract sample #.

FIG. 7 is a chart comprising a list of initially identified dysregulated protein dataset binned by gene ontology categories. The proteins annotated with either upward or downward facing arrows denote up- or down-regulation as assessed and validated by Western blotting analysis.

FIG. 8 is a Western Blot depicting changes in protein level regulation in hyperglycemia (HG) relative to normoglycemic conditions (NG). Laminin γ1 and Wnt16 expression levels are increased as a result of hyperglycemia, whereas ADAM15, and MMP-2 expression levels are decreased. The expression of Nemo kinase and ERK-12 are not affected by hyperglycemia.

FIG. 9, comprising FIG. 9A through FIG. 9C, is a series of charts depicting vulcano plots of the LC-MS peptide peaks. The left panel is a graph comparing hyperglycemia (HG) vs. control; The middle panel is a graph comparing HG-VEGF vs. HG; The right panel is a graph comparing HG-NOD vs. HG. The dots represent peaks that are either decreased (left of zero on the X axis) or increased (right of zero on the X axis). The light gray dots represent the 143 peaks that are significantly changed. The light gray dots above the horizontal dashed line following hyperglycemic insult represent the peaks that are significantly induced (right of the vertical dashed line on the right side of zero) and the decreased peaks are to the left of the vertical dashed line on the left side of zero). Note that in the VEGF-A165 and NOC-18 treated samples, the modulated peaks return to normoglycemic levels.

FIG. 10, comprising FIG. 10A through FIG. 10C, is a series of three micrographs illustrating arborization of the yolk sac vasculature in different glycemic conditions. FIG. 10A depicts the normal arborization of the yolk sac vasculature under normoglycemic (5 mM D-glucose) conditions. FIG. 10B depicts the arrest of yolk sac vascular development (failure of arborization) at the primary capillary plexus stage under hyperglycemic (20 mM D-glucose) conditions. FIG. 10C depicts the rescue of yolk Sac vascular development (normal arborization pattern) under under hyperglycemic (20 mM D-glucose) conditions plus the addition of either 2 pg rVEGF-A₁₆₅ or the slow release NO donor NOC-18.

FIG. 11, comprising FIG. 11A through FIG. 11E, is a series illustrations depicting the differential regulation of Wnt16 in yolk sacs across treatment groups and the affects of Wnt16, sequestration in the cardiac AVC assays. Validation of proteins identified during MS/MS runs were performed on yolk sacs treated with the four experimental conditions (Control, Hyperglycemis, Hyperglycemia+rVEGF-A₁₆₅ and Hyperglycemia+NOC-18). FIG. 11A depicts a Western blot, wherein Wnt16 was found to be upregulated by hyperglycemia but returned to normal levels by both VEGF and NO donor. FIG. 11A and FIG. 11E depict the AVC explant assay, wherein an antibody to Wnt16 blunted cardiac EMT resulting in a persistence of an epithelial phenotype compared to IgG control that displays a mesenchymal phenotype shown in FIG. 11B and FIG. 11D. B and C=phase contrast images; D and E=F-actin stained cultures.

FIG. 12, comprising FIG. 12A through FIG. 12E, is a series illustrations depicting the differential regulation of ADAM 15 in yolk sacs across treatment groups and the affects of ADAM 15 sequestration in cardiac AVC assays. Validation of proteins identified during MS/MS runs were performed on yolk sacs treated with the four experimental conditions (Control, Hyperglycemis, Hyperglycemia+rVEGF-A₁₆₅ and Hyperglycemia+NOC-18). FIG. 12A depicts a Western blot, wherein Adam15 was found to be downregulated by hyperglycemia but returned to normal levels by both VEGF and NO donor. FIG. 12C and FIG. 12E depict the AVC explant assay, wherein an antibody to the ectodomain of Adam15 blunted cardiac EMT, resulting in a persistence of an epithelial phenotype compared to IgG control that displays a mesenchymal phenotype shown in FIG. 12B and FIG. 12D. B and C=phase contrast images; D and E=F-actin stained cultures.

FIG. 13, comprising FIG. 13A through FIG. 13E, is a series illustrations depicting the differential regulation of NOGO A in yolk sacs across treatment groups and the affects of NOGO A sequestration in the cardiac AVC assays. Validation of proteins identified during MS/MS runs were performed on yolk sacs treated with the four experimental conditions (Control, Hyperglycemis, Hyperglycemia+rVEGF-A₁₆₅ and Hyperglycemia+NOC-18). FIG. 13A depicts a western blot, wherein NOGO-A was found to be downregulated by hyperglycemia but returned to normal levels by both VEGF and NO donor. FIG. 13C and FIG. 13E depict the AVC explant assay, wherein an antibody to the NOGO-A/B blunted cardiac EMT shown in FIG. 13F and FIG. 13G, resulting in a persistence of an epithelial phenotype compared to IgG control that displays a mesenchymal phenotype. B-E=phase contrast images.

FIG. 14, comprising FIG. 14A and FIG. 14B, is a series of images depicting the analyses of two selected factors in murine amniotic fluid from normal and diabetic pregnant mice. FIG. 14A depicts MMP-2 and FIG. 14B depicts and Laminin γ1 chain, identified during MS/MS runs on yolk sac lysates were assessed by Western blot in amniotic fluid samples.

FIG. 15, comprising FIG. 15A and FIG. 15B, depicts quantitation of data depicted in FIG. 14. FIG. 15A depicts a statistically significant decrease in MMP-2 and FIG. 15B depicts a statistically significant increase in Laminin in amniotic fluid of fetuses with cardiac anomalies compared to diabetic fetuses without cardiac anomalies or control fetuses from non-diabetic pregnancies.

FIG. 16, comprising a list comprising of the dysregulated protein dataset illustrating the identification of proteins associated with statistically significant peptide peaks. The 143 statistically significant peptide peaks that were dysregulated by hyperglycemia but returned to normal levels by both NO and VEGF, were targeted for MS/MS fragmentation in a second round of mass spectrometry. The subsequent spectra were compared against the NCBInr database using the MASCOT search engine to identify the proteins. Proteins were then mapped to gene ontology molecular function class. Of those listed the proteins marked by heavy arrows (Chondroitin 6-sulfotransferase, Protease, serine 3 and ST14) have been found to be dysregulated by hyperglycemic insult.

FIG. 17, comprising FIG. 17A and FIG. 17B, is a series of images depicting the analyses of Enolase, MMP-2 and MMP-9 in human amniotic fluid. FIG. 17A depicts the expression of Enolase, MMP-2 and MMP-9 in amniotic fluid as measured by Western Blot analysis from a small series of normal and diabetic pregnant women. FIG. 17A depicts decreased expression levels in the amniotic fluids obtained from diabetic women at 20 weeks of gestation. FIG. 17B illustrates that the reductions of MMP-2 and MMP-9 expression also were observed in a fluorescence enzymatic gelatinase assay.

FIG. 18, comprising FIG. 18A and FIG. 18B, is a series of images depicting the analyses of NG2 in human sera. FIG. 18A depicts Western Blots of serum NG2 levels wherein NG2 was found to be reduced in sera obtained from diabetic women at 20 weeks of gestation. FIG. 18B illustrates that the reduction in NG2 levels also were observed in a fluorescence enzymatic gelatinase assay.

FIG. 19, comprising FIG. 19A and FIG. 19B, is a series of images depicting expression of Laminin γ1 detected in the serum of diabetic women at 20 weeks of gestation. FIG. 19A depicts a Western Blot of Laminin γ1 expressed in serum wherein serum Lamininγ1 chain levels were found to be increased. FIG. 19B illustrates that the increase in Lamininγ levels also were observed in a fluorescence enzymatic gelatinase assay.

FIG. 20, comprising FIG. 20A through FIG. 20D, is a series of four charts illustrating the analyses of Western blot data obtained from 20 week gestation amniotic fluid samples from pregnant non-diabetic (dark squares) and women carrying fetuses with known congenital heart defects (CHD) (light squares). FIG. 20A illustrates the results for Wnt16; FIG. 20B depicts the results for PC1/3; FIG. 20C depicts results for SVCT. FIG. 20D depicts the result of plotting the results for two markers, PC1/3 and Wnt16 and illustrates separation of the CHD samples from controls (all controls fall within the circle).

FIG. 21, comprising a chart of the signaling pathways affected by hyperglycemia known to date. The proteomic dataset revealed proteins involved in migration/adhesion, differentiation, antioxidant defense, matrix remodeling, insulin/IGF homeostasis/signaling, protease activity and calcium regulation. Further, several proteins interact upstream or downstream of each other and with established factors in cardiovascular development. SVCT transports ascorbate (Vitamin C) into the cell which participates in matrix synthesis and antioxidant defense. Interestingly, dehydroascorbate enters the cell via glucose transporters (GLUT) prior to conversion to ascorbate. In the presence of glucose, dehydroascorbate is competitively inhibited from entering the cell which would comprise antioxidant defense. An additional antioxidant, NO, derived from eNOS/iNOS drives antioxidant defense and differentiation possibly via NO activation of TRPC5 which allows calcium to enter the cell. The influx of calcium activates the calcineurin/NFAT pathway which has complex interactions with DSCR and VEGF to drive differentiation. Additionally, jumonji may modulate this pathway at the level of VEGF. Wnt16 acting through canonical and non-canonical pathways affects β⁻-catenin and Snail levels in the nucleus, thus affecting differentiation. Leptin acting through Jak2, PI3K and Akt mediates differentiation. Akt seems to be a central molecule, modulating Leptin's actions, activating eNOS production of NO, driving MMP-2 activity and mediating the affects of IGF signaling. Cell surface proteoglycans such as Chondroitin Sulfate Proteoglycans participate in cell-matrix interactions and provide a sink for growth factors. Additional factors involved in adhesion/migration include integrin receptors such as αvβ3 integrin, which is a binding partner for ADAM15. ADAM15 proteolytic activity on cadherins could also affect differentiation and migration. Finally MMP-2 is another protease involved in migration and cytokine activation. Early in embryogenesis insulin is not present to direct growth instead fetal growth occurs via IGF signaling through Akt. Factors that regulate the receptor engagement or signaling cascade were identified on the extracellular face, including the proteases PC1/3 and ST14, and the intracellular face, including ZFP106 and HES6. Finally, crosstalk between leptin and insulin/IGF signaling may be mediated by the phosphatase PTPN1. This interaction may affect the etiology of obesity/diabetes as PTPN1 variants have been found in patients with obesity and diabetes. Collectively these data provide multiple avenues for future investigations of diabetes induced cardiovascular defects.

### DETAILED DESCRIPTION OF THE INVENTION

The present invention provides compositions and methods for the examination of maternal sera, amniotic fluid or the bodily fluids and tissues collectively known as body samples, to identify mothers who are at risk of experiencing hyperglycemia, who are at risk of developing gestational diabetes, and/or who are at risk of delivering offspring with congenital abnormalities as a result of maternal hyperglycemia.

The method of the invention comprises collecting a body sample from a pregnant woman. In preferred embodiments, the present invention uses multiplexed ELISAs which are more accurate than current tests (glycosylated HbA1c and glucose tolerance tests) in evaluating the state of the fetus at various time points of the pregnancy. The method of the invention comprises detecting elevated levels of at least one biomarker, wherein the over-expression of the biomarker specifically identifies samples indicative of a risk of fetal congenital defect or a woman at risk of experiencing hyperglycemia. The method of the invention also comprises detecting reduced levels of expression of at least one biomarker wherein the reduced expression of the biomarker specifically identifies samples indicative of a risk of fetal congenital defect.

The biomarkers of the invention are proteins and/or genes that are selectively over- or under-expressed during maternal hyperglycemia. Biomarkers of particular interest include matrix proteins, matrix metalloproteinsases, receptors and receptor ligands, proteins associated with cellular differentiation, transcriptional factors, apoptosis related proteins, cytoskeletal proteins, cell adhesion molecules, actin, mictrotubules, and proteins associated with glycemic metabolism and diabetes.

Over- or under-expression of any biomarker is assessed at the protein or nucleic acid level. In some embodiments of the invention immunohistochemistry techniques are provided that utilize antibodies to detect over- or under-expression of biomarkers in biological samples. Expression of biomarkers can also be detected by nucleic acid-based techniques, including, but not limited to, hybridization techniques and RT-PCR. Kits comprising reagents for practicing the methods of the invention are further provided.

The invention further includes a panel of biomarkers whose pattern of over- or under-expression is indicative of a fetus of a diabetic mother where the fetus is at risk of developing congenital malformations. The present invention also includes a panel of biomarkers that when over- or under-expressed can predict whether a pregnant woman is likely to experience hyperglycemia, or to develop gestational diabetes during pregnancy.

In addition, the invention includes the use of biomarker measurement to predict hyperglycemia in mammals at risk, although in a preferred embodiment, the mammal at risk is a pregnant female and its fetus.

### DEFINITIONS

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used in this specification and the appended claims, the singular forms “a”, “an”, and “the” include plural referents unless the content clearly dictates otherwise. Thus, for example, reference to “a pulmonary surfactant” includes a combination of two or more pulmonary surfactants, and the like.

An “amino acid” as used herein is meant to include both natural and synthetic amino acids, and both D and L amino acids. “Standard amino acid” means any of the twenty L-amino acids commonly found in naturally occurring peptides. “Nonstandard amino acid residues” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides, and particularly at the carboxy- or amino-terminus, can be modified by methylation, amidation, acetylation or substitution with other chemical groups which can change a peptide's circulating half life without adversely affecting activity of the peptide. Additionally, a disulfide linkage may be present or absent in the peptides.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426). As used herein, a “neutralizing antibody” is an immunoglobulin molecule that binds to and blocks the biological activity of the antigen.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The phrase “body sample” as used herein, is intended any sample comprising a cell, a tissue, or a bodily fluid in which expression of a biomarker can be detected. Examples of such body samples include but are not limited to blood, lymph, urine, gynecological fluids, biopsies, amniotic fluid and smears. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art.

The phrase “fetus at risk” as used herein refers to either a fetus with a greater than average likelihood of developing a congenital anomaly as a result of hyperglycemic insult experienced during gestation.

The phrase “woman (or mother) at risk” refers to a pregnant woman with a greater than average likelihood of experiencing hyperglycemia or of developing gestational diabetes during her pregnancy.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anti-codon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “diabetes” as used herein is defined as a metabolic disorder wherein pancreatic beta-cells of the animal, preferably human, produce little or no insulin, pancreatic beta-cells of the animal do not secrete insulin in response to glucose present in the bloodstream, or the animal's body is unable to use endogenous insulin to regulate blood glucose levels effectively. As a result, glucose concentration in the blood is elevated (hyperglycemia) and overflows into the urine. As a consequence the body loses its main source of fuel, despite high levels of glucose in the bloodstream.

The term “Type 1 diabetes” as used herein refers to an autoimmune disease wherein the immune system attacks and destroys the insulin-producing beta cells in the pancreas. The pancreas then produces little or no insulin. Generally, a person who has Type 1 diabetes must take insulin daily to survive.

The term “Type 2 diabetes” as used herein refers to the most common form of diabetes; 90 to 95 percent of people with diabetes have the Type 2 form of the disease. This form of diabetes is most often associated with older age, obesity, family history of diabetes, previous history of gestational diabetes, physical inactivity, and certain ethnicities. About 80 percent of people with Type 2 diabetes are overweight. When Type 2 diabetes is diagnosed, the pancreas is usually producing enough insulin, but for unknown reasons the body cannot use the insulin effectively, a condition called insulin resistance. After several years, insulin production decreases. The result is the same as for Type 1 diabetes—glucose builds up in the blood and the body cannot make efficient use of its main source of fuel.

The term “gestational diabetes” as used here refers to a metabolic condition some women develop late in pregnancy. Although this form of diabetes usually disappears after the birth of the baby, women who have had gestational diabetes have a 20 to 50 percent chance of developing Type 2 diabetes within 5 to 10 years.

The term “dysregulation” as used herein is used describes an over- or under-expression of a biomarker present and detected in a body sample obtained from a putative at-risk individual, then compared with a biomarker in a sample obtained from one or more normal, not-at-risk individuals. In some instances, the level of biomarker expression is compared with an average value obtained from more than one not-at-risk individuals. In other instances, the level of biomarker expression is compared with a biomarker level assessed in a sample obtained from one normal, not-at-risk sample. In yet another instance, the level of biomarker expression in the putative at-risk individual is compared with the level of biomarker expression in a sample obtained from the same individual at a different time.

A “putative at-risk individual” is a mammal, preferably a human, who is thought to be at risk of developing hyperglycemia.

The term “DNA” as used herein is defined as deoxyribonucleic acid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

As used herein, the term “fragment,” as applied to a protein or peptide, refers to a subsequence of a larger protein or peptide. A “fragment” of a protein or peptide can be at least about 20 amino acids in length; for example at least about 50 amino acids in length; at least about 100 amino acids in length, at least about 200 amino acids in length, at least about 300 amino acids in length, and at least about 400 amino acids in length (and any integer value in between).

The term “heterologous” as used herein is defined as DNA or RNA sequences or proteins that are derived from the different species.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. By way of example, the DNA sequences 3′ATTGCC5′ and 3′TATGGC are 50% homologous.

As used herein, an “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

By the term “specifically binds,” as used herein, is meant an antibody which recognizes and binds a biomarker or fragment thereof, but does not substantially recognize or bind other molecules in a sample.

The term “therapeutic” as used herein means a treatment and/or prophylaxis. A therapeutic effect is obtained by suppression; remission, or eradication of a disease state associated with liver disease.

The term “treatment” as used within the context of the present invention is meant to include therapeutic treatment as well as prophylactic, or suppressive measures for the disease or disorder. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

### DESCRIPTION

The present invention provides compositions and methods for identifying a pregnant woman whose fetus is at risk of developing a congenital defect as a result of some form of maternal hyperglycemia. The maternal hyperglycemia can occur as a single event that is sustained throughout the duration of the pregnancy, or it can occur episodically or as one or several events during pregnancy.

The present invention also includes compositions and methods of identifying a pregnant woman at risk of experiencing hyperglycemia or of developing gestational diabetes. In certain embodiments, the methods comprise the detection of over- and/or under-expression of specific biomarkers that are selectively dysregulated during maternal hyperglycemia. That is, detection of the level of the biomarkers of the current invention distinguishes between a pregnant woman who is normoglycemic and a pregnant woman who is hyperglycemic. Further, the present invention enables the practitioner to make an assessment as to whether or not the fetus is at risk for developing congenital malformations associated with hyperglycemia during pregnancy. Methods for detecting a fetus at risk of developing hyperglycemia associated congenital malformations involve the detection of the dysregulation of at least one biomarker in a body sample that is indicative of a potential congenital defect. In particular embodiments, antibodies and immunohistochemistry techniques are used to detect expression of the biomarkers of interest. In other embodiments, biomarker levels are detected by detecting nucleic acid levels. Kits for practicing the methods of the invention are further provided.

“Detecting a fetus at risk of developing congenital anomalies associated with maternal hyperglycemia” is intended to include, for example, diagnosing the presence of malformations of the central nervous system, the cardiovascular system, the gastrointestinal system, the urogenital system, the musculoskeletal system as well as other disorders associated with maternal hyperglycemia. It also is intended to include monitoring the fetus for the development or progression of said anomalies.

This invention is also useful in identifying mammals in general that are at risk for hyperglycemia. The level of biomarker expression on a body sample from the mammal, preferably a human, may be used to predict hyperglycemia in the mammal when assessed according to the methods disclosed herein.

### Biomarkers

Cardiovascular anomalies in babies born to hyperglycemic mothers are particularly devastating; these include transposition of the great vessels, ventricular septal defect, atrial septal defect, Tetralogy of Fallot, coarctation of the aorta, single umbilical artery, hypoplastic left heart and cardiomegaly. Several classes of proteins have been implicated as being important in cardiovascular development. These include, but are not limited to, matrix proteins, matrix metalloproteinases, receptors/receptor ligands, differentiation, transcription factors, apoptosis, cytoskeletal, cell adhesion, actin and mictotubules.

The biomarkers to be measured in the methods of the invention include genes and proteins, and variants and fragments thereof, that exhibit dysregulation in response to maternal hyperglycemia. Such biomarkers include DNA comprising the entire or partial sequence of the nucleic acid sequence encoding the biomarker, or the complement of such a sequence. Biomarker nucleic acids useful in the invention should be considered to include both DNA and RNA comprising the entire or partial sequence of any of the nucleic acid sequences of interest. A biomarker protein should be considered to comprise the entire or partial amino acid sequence of any of the biomarker proteins or polypeptides.

A “biomarker” is any gene, protein, or metabolite whose level of expression in a tissue, cell or bodily fluid is dysregulated compared to that of a normal or healthy cell, tissue, or biological fluid. Biomarkers to be measured in the methods of the invention selectively respond to maternal hyperglycemia or hyperglycemia in non-pregnant females or males.

By “selectively respond to maternal hyperglycemia” it is intended that the biomarker of interest is specifically over- or under-expressed in response to maternal hyperglycemia and is associated with either a fetus with a higher than normal risk for developing congenital malformations, a pregnant woman likely to experience hyperglycemia, and/or a pregnant woman more likely to develop gestational diabetes. This biomarker is not dysregulated during the course of a normal pregnancy, normal fetal development, or other conditions not considered to be clinical disease. Thus, measuring the levels of biomarkers in the methods of the invention permits differentiation between samples collected from a hyperglycemic mother carrying a fetus at risk of developing congenital defects as a result of maternal hyperglycemia from samples collected from a hyperglycemic mother carrying a fetus with no greater risk than average of congenital malformation. Further, by measuring the levels of the biomarkers in the method of the invention, a sample obtained from a pregnant woman at risk of experiencing hyperglycemia or developing gestational diabetes is differentiated from a sample obtained from a pregnant woman without greater risk of developing hyperglycemia and/or gestational diabetes.

The present invention also provides for analogs of polypeptides which comprise a biomarker protein. Analogs may differ from naturally occurring proteins or polypeptides by conservative amino acid sequence differences or by modifications which do not affect sequence, or by both. For example, conservative amino acid changes may be made, which although they alter the primary sequence of the protein or polypeptide, do not normally alter its function (e.g., secretion and capable of blocking virus infection). Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.
   Modifications (which do not normally alter primary sequence) include in vivo, or in vitro, chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

The present invention should also be construed to encompass “mutants,” “derivatives,” and “variants” of the biomarker proteins of the invention (or of the DNA encoding the same) which mutants, derivatives and variants are altered in one or more amino acids (or, when referring to the nucleotide sequence encoding the same, are altered in one or more base pairs) such that the resulting peptide (or DNA) is not identical to the sequences recited herein, but has the same biological property as the HBV surface proteins disclosed herein, in that the proteins have biological/biochemical properties. A biological property of the polypeptides of the present invention should be construed but not be limited to include, the ability to mediate normal cardiovascular embryogenesis.

Further, the invention should be construed to include naturally occurring variants or recombinantly derived mutants of biomarker proteins sequences, which variants or mutants render the polypeptide encoded thereby either more, less, or just as biologically active as wild type biomarker proteins.

The biological activity of the biomarkers of the invention is the ability of the biomarkers to respond in a predictable way to maternal hyperglycemia.

The measurement of biomarkers in the methods of the invention also distinguishes pathological changes in biomarker expression due to hyperglycemia from the normal pattern of expression of the same biomarkers observed during normal fetal development that are not indicative of clinical disease.

The biomarkers to be measured in the methods of the invention include any gene, protein, or metabolite that is selectively dysregulated by maternal hyperglycemia, as defined herein. Biomarkers of particular interest include genes and proteins involved in cardiac development, MET, and vasculogenesis. In some embodiments, the biomarker is an enzyme, a matrix protein, a matrix metalloproteinase, a receptor/ligand, a protein associated with cellular differentiation, a transcription factor, a protein associated with apoptosis, a cytoskeletal protein, a cell adhesion molecule, a protein associated with glucose metabolism or diabetes or a metabolite associated with glucose metabolism or diabetes. A nuclear biomarker may be used to practice certain aspects of the invention.

By “nuclear biomarker” is intended a biomarker that is predominantly expressed in the nucleus of the cell. A nuclear biomarker may be expressed to a lesser degree in other parts of the cell. Although any biomarker indicative of a fetus “at risk” for developing congenital malformations in response to hyperglycemia may be used in the present invention, in certain preferred embodiments the biomarkers are selected from the group consisting of laminin γ1 chain, laminin α4 chain, ADAM 15, MMP-2, MMP-9, Wnt16, enolase 1α, Down syndrome critical region protein, ST14, CH3T, PC1/3 and SVCT.

While not intending to be limited to any particular theory, in some embodiments, the molecular behavior of cardiac congenital malformation in response to maternal hyperglycemia can be characterized by the dysregulation of certain genes or proteins. Laminin γ1 and Wnt16 have both been shown to be up-regulated during maternal hyperglycemia, while ADAM 15, MMP-2, PC/1/3 and SVCT are down-regulated during maternal hyperglycemia as compared to normal controls.

The use of these molecular biomarkers, as compared to currently available techniques which test for maternal diabetes late in pregnancy, can improve the chance of detecting a fetus “at risk” of developing hyperglycemia related anomalies much earlier in pregnancy so that clinical intervention can be undertaken to abrogate or blunt the effects of the hyperglycemia. In particular aspects of the invention, the sensitivity and specificity of the present methods are equal to or greater than that of conventional detection techniques, i.e. glycosylated HbA1c and glucose tolerance tests in evaluating the state of the fetus at earlier time points than currently available.

As used herein, specificity refers to the level at which a method of the invention can accurately identify samples that have been confirmed as non-diabetic or not “at risk” (i.e., true negatives). That is, specificity is the proportion of disease negatives that are test-negative. In a clinical study, specificity is calculated by dividing the number of true negatives by the sum of true negatives and false positives.

By sensitivity is intended the level at which a method of the invention can accurately identify samples that have been confirmed as positive for hyperglycemia or have congenital defects (i.e., true positives). Thus, sensitivity is the proportion of disease positives that are test-positive. Sensitivity is calculated in a clinical study by dividing the number of true positives by the sum of true positives and false negatives. In some embodiments, the sensitivity of the disclosed methods for the detection of maternal hyperglycemia or an “at risk” fetus is preferably at least about 70%, more preferably at least about 80%, most preferably at least about 90, 91, 92, 93, 94, 95, 96, 97, 98, 99% or more. Furthermore, the specificity of the present methods is preferably at least about 70%, more preferably at least about 80%, most preferably at least about 90, 91, 92, 93, 94, 95, 96, 97, 98, 99% or more, and any and all Whole or partial integers in between.

The term positive predictive value or PPV refers to the probability that a patient has diabetes or is carrying a fetus “at risk” when restricted to those patients who are classified as positive using a method of the invention. PPV is calculated in a clinical study by dividing the number of true positives by the sum of true positives and false positives. In some embodiments, the PPV of a method of the invention for diagnosing maternal diabetes or a fetus “at risk” is at least about 40%, while maintaining a sensitivity of at least about 90% and more particularly at least about 95%. The “negative predictive value” or “NPV” of a test is the probability that the patient will not have the disease when restricted to all patients who test negative. NPV is calculated in a clinical study by dividing the number of true negatives by the sum of true negatives and false negatives.

Although the methods of the invention require the detection of at least one biomarker in a patient sample for the detection of a fetus or woman “at risk”, 2, 3, 4, 5, 6, 7, 8, 9, 10 or more biomarkers may be used to practice the method of the present invention. It is recognized that detection of more than one biomarker in a body sample may be necessary to identify instances of a pregnancy “at risk.” Therefore, in some embodiments, two or more biomarkers are used; more preferably, two or more complementary biomarkers are used.

By “complementary” when used to refer to a biomarker herein, is intended that detection of the combination of biomarkers in a body sample results in the successful identification of a fetus or woman “at risk” in a greater percentage of cases than would be identified if only one of the biomarkers was used. Thus, in some cases, a more accurate determination of a fetus “at risk” can be made by using at least two biomarkers. Accordingly, where at least two biomarkers are used, at least two antibodies directed to distinct biomarker proteins will be used to practice the immunocytochemistry methods disclosed herein. The antibodies may be contacted with the body sample simultaneously or concurrently.

In some embodiments, the methods for assessing fetal risk are performed as a reflex to a preexisting clinical situation, such as a woman diagnosed with Type 2 diabetes who becomes pregnant. In other aspects of the invention, the methods are performed as a primary screening test for women at risk of developing hyperglycemia during pregnancy.

### Detection

In particular embodiments, the diagnostic methods of the invention comprise collecting a sample from a patient, contacting the sample with at least one antibody specific for a biomarker of interest, and detecting antibody binding thereto. Samples that contain a dysregulated biomarker identify an individual at risk of experiencing hyperglycemia whether or not during pregnancy, an individual at risk of developing gestational diabetes, and/or a fetus at risk of developing a congenital anomaly as a result of maternal hyperglycemia.

Any methods available in the art for identification or detection of the biomarkers are encompassed herein. The dysregulation of a biomarker of the invention can be detected at a nucleic acid level or a protein level. In order to determine dysregulation of the biomarker, levels of the biomarker are measured in the body sample to be examined and compared with a corresponding body sample that originates from a normal, not-at-risk individual. In another embodiment of the invention, dysregulation of the biomarker is determined by measuring levels of the biomarker in the body sample to be examined and comparing with an average value obtained from more than one not-at-risk individuals. In still another embodiment of the invention, dysregulation of the biomarker is determined by measuring levels of the biomarker in the body sample to be examined and comparing with levels of biomarker obtained from a body sample obtained from the same individual at a different time,

Methods for detecting biomarkers of the invention comprise any method that determines the quantity or the presence of the biomarkers either at the nucleic acid or protein level. Such methods are well known in the art and include but are not limited to western blots, northern blots, southern blots, ELISA, immunoprecipitation, immunofluorescence, flow cytometry, immunocytochemistry, nucleic acid hybridization techniques, nucleic acid reverse transcription methods, and nucleic acid amplification methods. In particular embodiments, dysregulation of a biomarker is detected on a protein level using, for example, antibodies that are directed against specific biomarker proteins. These antibodies can be used in various methods such as Western blot, ELISA, immunoprecipitation, or immunocytochemistry techniques.

The invention should not be limited to any one method of protein or nucleic acid detection method recited herein, but rather should encompass all known or heretofor unknown methods of detection as are, or become, known in the art.

In one embodiment, antibodies specific for biomarker proteins are used to detect the dysregulation of a biomarker protein in a body sample. The method comprises obtaining a body sample from a patient, contacting the body sample with at least one antibody directed to a biomarker that is selectively dysregulated in maternal hyperglycemia to determine if the biomarker is dysregulated in the patient sample. One of skill in the art will recognize that the immunocytochemistry method described herein below is performed manually or in an automated fashion.

When the antibody used in the methods of the invention is a polygonal antibody (IgG), the antibody is generated by inoculating a suitable animal with a biomarker protein, peptide or a fragment thereof. Antibodies produced in the inoculated animal which specifically bind the biomarker protein are then isolated from fluid obtained from the animal. Biomarker antibodies may be generated in this manner in several non-human mammals such as, but not limited to goat, sheep, horse, rabbit, and donkey. Methods for generating polyclonal antibodies are well known in the art and are described, for example in Harlow, et al. (1988, In: Antibodies, A Laboratory Manual, Cold Spring Harbor, N.Y.). These methods are not repeated herein as they are commonly used in the art of antibody technology.

When the antibody used in the methods of the invention is a monoclonal antibody, the antibody is generated using any well known monoclonal antibody preparation procedures such as those described, for example, in Harlow et al. (supra) and in Tuszynski et al. (1988, Blood, 72:109-115). Given that these methods are well known in the art, they are not replicated herein. Generally, monoclonal antibodies directed against a desired antigen are generated from mice immunized with the antigen using standard procedures as referenced herein. Monoclonal antibodies directed against full length or peptide fragments of biomarker may be prepared using the techniques described in Harlow, et al. (1988, In: Antibodies, A Laboratory Manual, Cold Spring Harbor, N.Y.).

Samples may need to be modified in order to render the biomarker antigens accessible to antibody binding. In a particular aspect of the immunocytochemistry methods, slides are transferred to a pretreatment buffer, for example phosphate buffered saline containing Triton-X. Incubating the sample in the pretreatment buffer rapidly disrupts the lipid bilayer of the cells and renders the antigens (i.e., biomarker proteins) more accessible for antibody binding. The pretreatment buffer may comprise a polymer, a detergent, or a nonionic or anionic surfactant such as, for example, an ethyloxylated anionic or nonionic surfactant, an alkanoate or an alkoxylate or even blends of these surfactants or even the use of a bile salt. The pretreatment buffers of the invention are used in methods for making antigens more accessible for antibody binding in an immunoassay, such as, for example, an immunocytochemistry method or an immunohistochemistry method.

Any method for making antigens more accessible for antibody binding may be used in the practice of the invention, including antigen retrieval methods known in the art. See, for example, Bibbo, 2002, Acta. Cytol. 46:25 29; Saqi, 2003, Diagn. Cytopathol. 27:365 370; Bibbo, 2003, Anal. Quant. Cytol. Histol. 25:8 11. In some embodiments, antigen retrieval comprises storing the slides in 95% ethanol for at least 24 hours, immersing the slides one time in Target Retrieval Solution pH 6.0 (DAKO S1699)/dH2O bath preheated to 95° C., and placing the slides in a steamer for 25 minutes.

Following pretreatment or antigen retrieval to increase antigen accessibility, samples are blocked using an appropriate blocking agent, e.g., a peroxidase blocking reagent such as hydrogen peroxide. In some embodiments, the samples are blocked using a protein blocking reagent to prevent non-specific binding of the antibody. The protein blocking reagent may comprise, for example, purified casein, serum or solution of milk proteins. An antibody directed to a biomarker of interest is then incubated with the sample.

As noted elsewhere herein, one of skill in the art will appreciate that a more accurate diagnosis of a pregnant woman at risk of developing hyperglycemia, or a woman at risk of developing gestational diabetes, or a fetus at risk for developing congenital malformation as a result of maternal hyperglycemia may be obtained in some cases by detecting more than one biomarker in a patient sample. Therefore, in particular embodiments, at least two antibodies directed to two distinct biomarkers are used. Where more than one antibody is used, these antibodies may be added to a single sample sequentially as individual antibody reagents or simultaneously as an antibody cocktail. Alternatively, each individual antibody may be added to a separate sample from the same patient, and the resulting data pooled.

Techniques for detecting antibody binding are well known in the art. Antibody binding to a biomarker of interest may be detected through the use of chemical reagents that generate a detectable signal that corresponds to the level of antibody binding and, accordingly, to the level of biomarker protein expression. In one of the preferred immunocytochemistry methods of the invention, antibody binding is detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell staining that corresponds to expression level of the biomarker of interest. Enzymes of particular interest include horseradish peroxidase (HRP) and alkaline phosphatase (AP). Commercial antibody detection systems, such as, for example the Dako Envision+ system (Dako North America, Inc., Carpinteria, Calif.) and Mach 3 system (Biocare Medical, Walnut Creek, Calif.), may be used to practice the present invention.

In one particular immunocytochemistry method of the invention, antibody binding to a biomarker is detected through the use of an HRP-labeled polymer that is conjugated to a secondary antibody. Antibody binding can also be detected through the use of a mouse probe reagent, which binds to mouse monoclonal antibodies, and a polymer conjugated to HRP, which binds to the mouse probe reagent. Slides are stained for antibody binding using the chromogen 3,3-diaminobenzidine (DAB) and then counterstained with hematoxylin and, optionally, a bluing agent such as ammonium hydroxide or TBS/Tween-20. In some aspects of the invention, slides are reviewed microscopically by a cytotechnologist and/or a pathologist to assess cell staining (i.e., biomarker overexpression). Alternatively, samples may be reviewed via automated microscopy or by personnel with the assistance of computer software that facilitates the identification of positive staining cells.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, 131I, 35S, or ³H.

In regard to detection of antibody staining in the immunocytochemistry methods of the invention, there also exist in the art video-microscopy and software methods for the quantitative determination of an amount of multiple molecular species (e.g., biomarker proteins) in a biological sample, wherein each molecular species present is indicated by a representative dye marker having a specific color. Such methods are also known in the art as calorimetric analysis methods. In these methods, video-microscopy is used to provide an image of the biological sample after it has been stained to visually indicate the presence of a particular biomarker of interest. Some of these methods, such as those disclosed in U.S. patent application Ser. No. 09/957,446 and U.S. patent application Ser. No. 10/057,729 to Marcelpoil., incorporated herein by reference, disclose the use of an imaging system and associated software to determine the relative amounts of each molecular species present based on the presence of representative color dye markers as indicated by those color dye markers' optical density or transmittance value, respectively, as determined by an imaging system and associated software. These techniques provide quantitative determinations of the relative amounts of each molecular species in a stained biological sample using a single video image that is “deconstructed” into its component color parts.

The antibodies used to practice the invention are selected to have high specificity for the biomarker proteins of interest. Methods for making antibodies and for selecting appropriate antibodies are known in the art. See, for example, Celis, J. E. ed. (in press) Cell Biology & Laboratory Handbook, 3rd edition (Academic Press, New York), which is herein incorporated in its entirety by reference. In some embodiments, commercial antibodies directed to specific biomarker proteins may be used to practice the invention. The antibodies of the invention may be selected on the basis of desirable staining of cytological, rather than histological, samples. That is, in particular embodiments the antibodies are selected with the end sample type (i.e., cytology preparations) in mind and for binding specificity.

One of skill in the art will recognize that optimization of antibody titer and detection chemistry is needed to maximize the signal to noise ratio for a particular antibody. Antibody concentrations that maximize specific binding to the biomarkers of the invention and minimize non-specific binding (or “background”) will be determined in reference to the type of biological sample being tested. In particular embodiments, appropriate antibody titers for use cytology preparations are determined by initially testing various antibody dilutions on formalin-fixed paraffin-embedded normal tissue samples. Optimal antibody concentrations and detection chemistry conditions are first determined for formalin-fixed paraffin-embedded tissue samples. The design of assays to optimize antibody titer and detection conditions is standard and well within the routine capabilities of those of ordinary skill in the art. After the optimal conditions for fixed tissue samples are determined, each antibody is then used in cytology preparations under the same conditions. Some antibodies require additional optimization to reduce background staining and/or to increase specificity and sensitivity of staining in the cytology samples.

Furthermore, one of skill in the art will recognize that the concentration of a particular antibody used to practice the methods of the invention will vary depending on such factors as time for binding, level of specificity of the antibody for the biomarker protein, and method of body sample preparation. Moreover, when multiple antibodies are used, the required concentration may be affected by the order in which the antibodies are applied to the sample, i.e., simultaneously as a cocktail or sequentially as individual antibody reagents. Furthermore, the detection chemistry used to visualize antibody binding to a biomarker of interest must also be optimized to produce the desired signal to noise ratio.

As noted, it is contemplated that the biomarkers of the invention will find utility as immunogens, e.g., in immunohistochemistry and in ELISA assays. One evident utility of the encoded antigens and corresponding antibodies is in immunoassays for the detection of biomarker proteins, as needed in diagnosis and prognostic monitoring.

### Immunoassays

Immunoassays, in their simplest and most direct sense, are binding assays. Certain preferred immunoassays are the various types of enzyme linked immunosorbent assays (ELISA) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, antibodies binding to the biomarker proteins of the invention are immobilized onto a selected surface exhibiting protein affinity, such as a well in a polystyrene microtiter plate. Then, a test composition suspected of containing the biomarker antigen, such as a clinical sample, is added to the wells. After binding and washing to remove non-specifically bound immunecomplexes, the bound antibody may be detected. Detection is generally achieved by the addition of a second antibody specific for the target protein, that is linked to a detectable label. This type of ELISA is a simple “sandwich ELISA”. Detection may also be achieved by the addition of a second antibody, followed by the addition of a third antibody that has binding affinity for the second antibody, with the third antibody being linked to a detectable label.

In another exemplary ELISA, the samples suspected of containing the biomarker antigen are immobilized onto the well surface and then contacted with the antibodies of the invention. After binding and washing to remove non-specifically bound immunecomplexes, the bound antigen is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Another ELISA in which the proteins or peptides are immobilized, involves the use of antibody competition in the detection. In this ELISA, labeled antibodies are added to the wells, allowed to bind to the biomarker protein, and detected by means of their label. The amount of marker antigen in an unknown sample is then determined by mixing the sample with the labeled antibodies before or during incubation with coated wells. The presence of marker antigen in the sample acts to reduce the amount of antibody available for binding to the well and thus reduces the ultimate signal. This is appropriate for detecting antibodies in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immunecomplexes. These are described as follows:

In coating a plate with either antigen or antibody, the wells of the plate are incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating of nonspecific adsorption sites on the immobilizing surface reduces the background caused by nonspecific binding of antisera to the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control and/or clinical or biological sample to be tested under conditions effective to allow immunecomplex (antigen/antibody) formation. Detection of the immunecomplex then requires a labeled secondary binding ligand or antibody, or a secondary binding ligand or antibody in conjunction with a labeled tertiary antibody or third binding ligand.

“Under conditions effective to allow immunecomplex (antigen/antibody) formation” means that the conditions preferably include diluting the antigens and antibodies with solutions such as, but not limited to, BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hours, at temperatures preferably on the order of 25° to 27° C., or may be overnight at about 4° C.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this label is an enzyme that generates a color or other detectable signal upon incubating with an appropriate chromogenic or other substrate. Thus, for example, the first or second immunecomplex can be detected with a urease, glucose oxidase, alkaline phosphatase or hydrogen peroxidase-conjugated antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation is then achieved by measuring the degree of color generation, e.g., using a visible spectra spectrophotometer.

### Nucleic Acid-Based Techniques

In other embodiments, the expression of a biomarker of interest is detected at the nucleic acid level. Nucleic acid-based techniques for assessing expression are well known in the art and include, for example, determining the level of biomarker mRNA in a body sample. Many expression detection methods use isolated RNA. Any RNA isolation technique that does not select against the isolation of mRNA can be utilized for the purification of RNA from body samples (see, e.g., Ausubel, ed., 1999, Current Protocols in Molecular Biology (John Wiley & Sons, New York). Additionally, large numbers of tissue samples can readily be processed using techniques well known to those of skill in the art, such as, for example, the single-step RNA isolation process of Chomczynski, 1989, U.S. Pat. No. 4,843,155).

The term “probe” refers to any molecule that is capable of selectively binding to a specifically intended target biomolecule, for example, a nucleotide transcript or a protein encoded by or corresponding to a biomarker. Probes can be synthesized by one of skill in the art, or derived from appropriate biological preparations. Probes may be specifically designed to be labeled with a detectable label. Examples of molecules that can be used as probes include, but are not limited to, RNA, DNA, proteins, antibodies, and organic molecules.

Isolated mRNA as a biomarker can be detected in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a biomarker of the present invention. Hybridization of an mRNA with the probe indicates that the biomarker in question is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array (Santa Clara, Calif.). A skilled artisan can readily adapt known mRNA detection methods for use in detecting the level of mRNA encoded by the biomarkers of the present invention.

An alternative method for determining the level of biomarker mRNA in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189 193), self sustained sequence replication (Guatelli, 1990, Proc. Natl. Acad. Sci. USA, 87:1874 1878), transcriptional amplification system (Kwoh, 1989, Proc. Natl. Acad. Sci. USA, 86:1173 1177), Q-Beta Replicase (Lizardi, 1988, Bio/Technology, 6:1197), rolling circle replication (Lizardi, U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, biomarker expression is assessed by quantitative fluorogenic RT-PCR (i.e., the TaqMan® System). Such methods typically use pairs of oligonucleotide primers that are specific for the biomarker of interest. Methods for designing oligonucleotide primers specific for a known sequence are well known in the art.

Biomarker expression levels of RNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of biomarker expression may also comprise using nucleic acid probes in solution.

### Microarray

In one embodiment of the invention, microarrays are used to detect biomarker expression in biological samples. Microarrays are particularly well suited for this purpose because of the reproducibility between trials. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

Techniques for the synthesis of these arrays using mechanical synthesis methods are described in, e.g., U.S. Pat. No. 5,384,261, incorporated herein by reference in its entirety for all purposes. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. Arrays may be packaged in such a manner as to allow for diagnostics or other manipulation of an all-inclusive device. See, for example, U.S. Pat. Nos. 5,856,174 and 5,922,591 herein incorporated by reference.

Nucleic acids which code for the biomarkers can be placed in an array on a substrate, such as on a chip (e.g., DNA chip or microchips). These arrays also can be placed on other substrates, such as microtiter plates, beads or microspheres. Methods of linking nucleic acids to suitable substrates and the substrates themselves are described, for example, in U.S. Pat. Nos. 5,981,956; 5,922,591; 5,994,068 (Gene Logic's Flow-thru ChipO Probe ArraysO); U.S. Pat. Nos. 5,858,659, 5,753,439; 5,837,860 and the FlowMetrix technology (e.g., microspheres) of Luminex (U.S. Pat. Nos. 5,981,180 and 5,736,330).

There are two preferred methods to make a nucleic acid array. One is to synthesize the specific oligonucleotide sequences directly onto the solid-phase in the desired pattern (Southern, 1994, Nucl. Acids Res., 22: 1368-73; Maskos, 1992, Nucl. Acids Res., 20: 1679-84; Pease, 1994, Proc. Natl. Acad. Sci., 91: 5022-6; and U.S. Pat. No. 5,837,860) and the other is to presynthesize the oligonucleotides in an automated DNA synthesizer and then attach the oligonucleotides onto the solid-phase support at specific locations (Lamture, 1994, Nucl. Acids Res., 22: 2121; Smith, 1994, Nucl. Acids Res., 22: 5456 64. In the first method, the efficiency of the coupling step of each base affects the quality and integrity of the nucleic acid molecule array.

A second, more preferred method for nucleic acid array synthesis utilizes an automated DNA synthesizer for DNA synthesis. The controlled chemistry of an automated DNA synthesizer allows for the synthesis of longer, higher quality DNA molecules than is possible with the first method. Also, the nucleic acid molecules synthesized can be purified prior to the coupling step. The nucleic acids can be attached to the substrate as described in U.S. Pat. No. 5,837,860.

Thus, for example, covalently immobilized nucleic acid molecules may be used to detect specific PCR products by hybridization where the capture probe is immobilized on the solid phase or substrate (Ranki, 1983, Gene, 21: 77-85; Keller, 1991, Clin. Microbiol., 29: 638-41; Urdea, 1987, Gene, 61: 253-64). A preferred method would be to prepare a single-stranded PCR product before hybridization. A patient sample that is suspected to contain the biomarker molecule, or an amplification product thereof, would then be exposed to the solid-surface and permitted to hybridize to the bound oligonucleotide.

The methods of the present invention do not require that the target nucleic acid contain only one of its natural two strands. Thus, the methods of the present invention may be practiced on either double-stranded DNA (dsDNA), or on single-stranded DNA (ssDNA) obtained by, for example, alkali treatment of native DNA. The presence of the unused (non-template) strand does not affect the reaction.

Where desired, however, any of a variety of methods can be used to eliminate one of the two natural stands of the target DNA molecule from the reaction. Single-stranded DNA molecules may be produced using the ssDNA bacteriophage, M13 (Messing, 1983, Meth. Enzymol., 101: 20-78; see also, Sambrook, 2001, Molecular Cloning: A Laboratory Manuel. 3^{rd} ed. (Cold Spring Harbor Laboratory Press, Cold Spring Harbor, N.Y.).

Several alternative methods can be used to generate single-stranded DNA molecules. For example, Gyllensten, 1988, Proc. Natl. Acad. Sci. U.S.A., 85: 7652-6 and Mihovilovic, 1989, BioTechiques, 7: 14-6 describe a method, termed “asymmetric PCR,” in which the standard “PCR” method is conducted using primers that are present in different molar concentrations.

Other methods have also exploited the nuclease resistant properties of phosphorothioate derivatives in order to generate single-stranded DNA molecules (U.S. Pat. No. 4,521,509; Sayers, 1988, Nucl. Acids Res., 16: 791-802; Eckstein, 1976, Biochemistry 15: 1685-91; Ott, 1987, Biochemistry 26: 8237-41; see also, Sambrook, 2001, Molecular Cloning: A Laboratory Manuel, 3^{rd} ed. (Cold Spring Harbor Laboratory Press, Cold Spring Harbor, N.Y.).

Screening for multiple genes in samples of genomic material according to the methods of the present invention, is generally carried out using arrays of oligonucleotide probes. These arrays may generally be “tiled” for a large number of specific genes. By “tiling” is generally meant the synthesis of a defined set of oligonucleotide probes which is made up of a sequence complementary to the target sequence of interest, as well as pre-selected variations of that sequence, e.g., substitution of one or more given positions with one or more members of the basic set of monomers. i.e. nucleotides. Tiling strategies are discussed in detail in Published PCT Application No. WO 95/11995, incorporated herein by reference in its entirety for all purposes. By “target sequence” is meant a sequence which has been identified as encoding a biomarker of interest or portion thereof, a related polymorphism or mutation (e.g., a single-base polymorphism also referred to as a “biallelic base”) of one of the identified biomarkers. It will be understood that the term “target sequence” is intended to encompass the various forms present in a particular sample of genomic material, i.e., both alleles in a diploid genome.

In a particular aspect, arrays are tiled for a number of specific, identified biomarker sequences. In particular, the array is tiled to include a number of detection blocks, each detection block being specific for a particular biomarker or set of biomarkers. For example, a detection block may be tiled to include a number of probes which span the sequence segment that includes a specific biomarker or a polymorphism thereof. To ensure probes that are complementary to each variant, the probes are synthesized in pairs differing, for example, at the biallelic base.

In addition to the probes differing at the biallelic bases, monosubstituted probes can be generally tiled within the detection block. These monosubstituted probes have up to a certain number of bases in either direction from the polymorphisms, substituted with the remaining nucleotides (selected from A, T, G, C or U). Typically, the probes in a tiled detection block will include substitutions of the sequence positions up to and including those that are 5 bases away from the base that corresponds to the polymorphism. Preferably, bases up to and including those in positions 2 bases from the polymorphism will he substituted. The monosubstituted probes provide internal controls for the tiled array, to distinguish actual hybridization from artifactual cross-hybridization.

A variety of tiling configurations may also be employed to ensure optimal discrimination of perfectly hybridizing probes. For example, a detection block may be tiled to provide probes having optimal hybridization intensities with minimal cross-hybridization. For example, where a sequence downstream from a polymorphic base is G C rich, it could potentially give rise to a higher level of cross-hybridization or “noise,” when analyzed. Accordingly, one can tile the detection block to take advantage of more of the upstream sequence.

Optimal tiling configurations may be determined for any particular biomarker or polymorphism by comparative analysis. For example, triplet or larger detection blocks may be readily employed to select such optimal tiling strategies.

Additionally, arrays will generally be tiled to provide for ease of reading and analysis. For example, the probes tiled within a detection block will generally be arranged so that reading across a detection block the probes are tiled in succession, i.e., progressing along the target sequence one or more nucleotides at a time.

Once an array is appropriately tiled for a given biomarker and related polymorphism or set of polymorphisms, the target nucleic acid is hybridized with the array and scanned. A target nucleic acid sequence, which includes one or more previously identified biomarkers, is amplified by well known amplification techniques, e.g., polymerase chain reaction (PCR). Typically, this involves the use of primer sequences that are complementary to the two strands of the target sequence both upstream and downstream from the polymorphism. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single biomarker, in the preferred aspects, the arrays of the invention will include multiple detection blocks, and thus be capable of analyzing multiple, specific biomarkers. For example, preferred arrays will generally include from about 50 to about 4,000 different detection blocks with particularly preferred arrays including from 10 to 3,000 different detection blocks.

In alternate arrangements, it will generally be understood that detection blocks may be grouped within a single array or in multiple, separate arrays so that varying, optimal conditions may be used during the hybridization of the target to the array. For example, it may often be desirable to provide for the detection of those polymorphisms that fall within G C rich stretches of a genomic sequence, separately from those falling in A T rich segments. This allows for the separate optimization of hybridization conditions for each situation.

In one approach, total mRNA isolated from the sample is converted to labeled cRNA and then hybridized to an oligonucleotide array. Each sample is hybridized to a separate array. Relative transcript levels may be calculated by reference to appropriate controls present on the array and in the sample.

### Reporter Genes and Tags

In order to assess the expression of a biomarker proteins or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (e.g., Ui-Tei, 2000, FEBS Letters, 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### Preparation of Nucleic Acid Probes

Using the sequence information provided herein, the nucleic acids may be synthesized according to a number of standard methods known in the art. Oligonucleotide synthesis, is carried out on commercially available solid phase oligonucleotide synthesis machines or manually synthesized using the solid phase phosphoramidite triester method described by Beaucage, 1981, Tetrahedron Letters, 22: 1859-1862.

Once a nucleic acid encoding a biomarker is synthesized, it may be amplified and/or cloned according to standard methods in order to produce recombinant polypeptides. Molecular cloning techniques to achieve these ends are known in the art. A wide variety of cloning and in vitro amplification methods suitable for the construction of recombinant nucleic acids are known to those skilled in the art.

Examples of techniques sufficient to direct persons of skill through in vitro amplification methods, including the polymerase chain reaction (PCR), the ligase chain reaction (LCR), and other DNA or RNA polymerase-mediated techniques are found in Sambrook, 2001, Molecular Cloning: A Laboratory Manuel, 3^{rd} ed. (Cold Spring Harbor Laboratory Press, Cold Spring Harbor, N.Y.

Once the nucleic acid for a biomarker is cloned, a skilled artisan may express the recombinant gene(s) in a variety of engineered cells. Examples of such cells include bacteria, yeast, filamentous fungi, insect (especially employing baculoviral vectors), and mammalian cells. It is expected that those of skill in the art are knowledgeable in the numerous expression systems available for expressing the biomarker proteins of the invention.

### Kits

Kits for practicing the methods of the invention are further provided. By “kit” is intended any manufacture (e.g., a package or a container) comprising at least one reagent, e.g., an antibody, a nucleic acid probe, etc. for specifically detecting the expression of a biomarker of the invention. The kit may be promoted, distributed, or sold as a unit for performing the methods of the present invention. Additionally, the kits may contain a package insert describing the kit and including instructional material for its use.

In a particular embodiment, kits for practicing the immunocytochemistry methods of the invention are provided. Such kits are compatible with both manual and automated immunocytochemistry techniques (e.g., cell staining). These kits comprise at least one antibody directed to a biomarker of interest, chemicals for the detection of antibody binding to the biomarker, a counterstain, and, optionally, a bluing agent to facilitate identification of positive staining cells. Any chemicals that detect antigen-antibody binding may be used in the practice of the invention. In some embodiments, the detection chemicals comprise a labeled polymer conjugated to a secondary antibody. For example, a secondary antibody that is conjugated to an enzyme that catalyzes the deposition of a chromogen at the antigen-antibody binding site may be provided. Such enzymes and techniques for using them in the detection of antibody binding are well known in the art. In one embodiment, the kit comprises a secondary antibody that is conjugated to an HRP-labeled polymer. Chromogens compatible with the conjugated enzyme (e.g., DAB in the case of an HRP-labeled secondary antibody) and solutions, such as hydrogen peroxide, for blocking non-specific staining may be further provided. In other embodiments, antibody binding to a biomarker protein is detected through the use of a mouse probe reagent that binds to mouse monoclonal antibodies, followed by addition of a dextran polymer conjugated with HRP that binds to the mouse probe reagent. Such detection reagents are commercially available from, for example, Biocare Medical.

The kits of the present invention may further comprise a peroxidase blocking reagent (e.g., hydrogen peroxide), a protein blocking reagent (e.g., purified casein), and a counterstain (e.g., hematoxylin). A bluing agent (e.g., ammonium hydroxide or TBS, pH 7.4, with Tween-20 and sodium azide) may be further provided in the kit to facilitate detection of positive staining cells.

In another embodiment, the immunocytochemistry kits of the invention additionally comprise at least two reagents, e.g., antibodies, for specifically detecting the expression of at least two distinct biomarkers. Each antibody may be provided in the kit as an individual reagent or, alternatively, as an antibody cocktail comprising all of the antibodies directed to the different biomarkers of interest. Furthermore, any or all of the kit reagents may be provided within containers that protect them from the external environment, such as in sealed containers.

Positive and/or negative controls may be included in the kits to validate the activity and correct usage of reagents employed in accordance with the invention. Controls may include samples, such as tissue sections, cells fixed on glass slides, etc., known to be either positive or negative for the presence of the biomarker of interest. The design and use of controls is standard and well within the routine capabilities of those of ordinary skill in the art.

One of skill in the art will further appreciate that any or all steps in the methods of the invention could be implemented by personnel or, alternatively, performed in an automated fashion. Thus, the steps of body sample preparation, sample staining, and detection of biomarker expression may be automated.

### Identification of Biomarkers

Human and animal plasma and serum represents an important biological material for disease diagnosis and the presence of large number of diagnostically important proteins makes plasma an excellent starting material for biomarker discovery. However, it also represents a tremendous analytical challenge because the estimated dynamic range of protein concentrations in human serum can be up to 12 orders of magnitude. (Anderson and Anderson, 2002, The human plasma proteome: History, character, and diagnostic prospects. Mol. Cell. Proteomics, 1: 845-867). Innovations in both sample preparation and protein analysis are therefore necessary to push the analytical capabilities towards the 10¹² required range. To study the low abundance fraction of the serum proteome it has become critical to use various depletion strategies.

The present invention encompasses a high-throughput method of identifying proteins that are dysregulated by maternal hyperglycemia. Quantitative proteomics requires the analysis of complex protein samples. In the case of clinical diagnosis, the ability to obtain appropriate specimens for clinical analysis is important for ease and accuracy of diagnosis. A number of biologically important molecules are secreted and are therefore present in body fluids such as blood and serum, cerebrospinal fluid, saliva, urine, amniotic fluid, and the like. In addition to the presence of important biological molecules, body fluids also provide an attractive specimen source because body fluids are generally readily accessible and available in reasonable quantities for clinical analysis. It is therefore apparent that a general method for the quantitative analysis of the proteins contained in body fluids in health and disease would be of great diagnostic and clinical importance.

A key problem with the proteomic analysis of serum and many other body fluids is the peculiar protein composition of these specimens. The protein composition is dominated by a few proteins that are extraordinarily abundant, with albumin alone representing 50% of the total plasma proteins. Due to the abundance of these major proteins as well as the presence of multiple modified forms of these abundant proteins, the large number of protein species of lower abundance are obscured or inaccessible by traditional proteomics analysis methods such as two-dimensional electrophoresis (2DE).

In the present invention, albumin depletion of serum is accomplished using the Qproteome Murine albumin depletion kit (Qiagen, Valencia, Calif.) and alpha-fetoprotein is depleted from amniotic fluid. Additionally, the ProteoPrep 20 Plasma Immunodepletion kit (Sigma, St. Louis, Mo.) is used to remove abundant proteins from the samples. This new sample preparation technology depletes highly abundant proteins, thus enhancing detection of low copy number proteins in biofluids using mass spectrometry. Candidate proteins in amniotic fluid and maternal serum for predicting clinical outcomes linked to hyperglycemia in pregnancy are derived via proteomic analysis from the culture and animal models described as well as material obtained from human samples.

Although the methods of the invention are advantageous in that complex biological samples can be analyzed directly, a sample can also be processed, if desired prior to analysis. For example, a blood or amniotic fluid sample can be fractionated to isolate particular cell types, for example, red blood cells, white blood cells, fetal cells, stem cells, and the like. A sample can also be fractionated to isolate particular types of proteins, for example, based on structural or functional properties such as serum proteins modified by glycosylation, phosphorylation, or other post-translational modifications, or proteins having a particular affinity, such as an affinity for nucleic acids. A serum sample can also be fractionated based on physical-chemical properties, for example, size, pI, and the like. A serum sample can additionally be fractionated to remove bulk proteins present in large quantities, such as albumin, to facilitate analysis of less abundant serum polypepties. Furthermore, a cellular sample can be fractionated to isolate subcellular organelles. Moreover, a cellular or tissue sample can be solubilized and fractionated by any of the well known fractionation methods, including chromatographic techniques such as ion exchange, hydrophobic and reverse phase, size exclusion, affinity, hydrophobic charge-induction chromatography, and the like (Ausubel, 1993, Scopes, Protein Purification: Principles and Practice, third edition, Springer-Verlag, New York; Burton and Harding, 1998, J Chromatogr, A 814:71-81).

Although the methods of the invention are particularly useful for analyzing complex samples such as biological samples, the methods can also be used on samples of reduced complexity. For example, the sample can be fractionated, as described above, to provide a smaller number of sample molecules to be captured on solid phase, including prior affinity chromatography. In addition, the sample can be a highly purified sample, including essentially a single purified molecule such as a polypeptide or nucleic acid or molecule that is expressed at high levels in the sample, for example, by recombinant methods.

If desired, the sample may be separated or fractionated by a number of known fractionation techniques. Fractionation techniques can be applied to the samples at any of a number of suitable points in the methods of the invention. For example, a sample can be fractionated prior to binding to a solid support. Thus if desired a substantially purified fraction of protein is available for immobilization to a chip or other support matrix. One skilled in the art can readily determine appropriate steps for fractionating sample molecules based on the needs of the particular application of methods of the invention.

Methods for fractionating sample molecules are well known to those skilled in the art. These separation methods include, but are not limited to, liquid chromatography where the components are separated by their differences in hydrophobicity, or electrophoresis, particularly slab gel or capillary electrophoresis. The electrophoresis may involve one- or two-dimensional electrophoresis, may be in a gel, may use a capillary or may use a channel in a microfluidic device. (See e.g., Opiteck, 1998, Anal. Chem., 258:349-61; U.S. Pat. Nos. 4,415,655; 4,481,094; 4,865,707; and 4,946,794; Laemmli, 1970, U K, Nature, 227; 680-685; and see also, Sambrook, 2001, Molecular Cloning: A Laboratory Manuel, 3^{rd} ed. (Cold Spring Harbor Laboratory Press, Cold Spring Harbor, N.Y.). Liquid chromatography may use a combination of size exclusion liquid chromatography followed by RP-HPLC or only RP-HPLC. The conditions employed are conventional for liquid chromatographic separation of proteins and peptides and commercial equipment and materials are available. See, e.g., U.S. Pat. Nos. 5,041,538 and 5,290,920 and WO91/15228, as exemplary. A suitable eluant can include a water/acetonitrile gradient, optionally containing 0.1% trifluoroacetic acid; 0.1% trifluoroacetic acid; or 0.1% formic acid. The conjugates can be monitored by their fluorescence and may be isolated in wells for further investigation. A separation profile in such methods may be sufficient information to identify the peptide and, therefore, the protein.

The terms “mass spectrometry” or “MS” as used herein refer to methods of filtering, detecting, and measuring ions based on their mass-to-charge ratio, or “m/z.” In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). See U.S. Pat. No. 6,204,500, entitled “Mass Spectrometry From Surfaces;” U.S. Pat. No. 6,107,623, entitled “Methods and Apparatus for Tandem Mass Spectrometry;” U.S. Pat. No. 6,268,144, entitled “DNA Diagnostics Based On Mass Spectrometry;” U.S. Pat. No. 6,124,137, entitled “Surface-Enhanced Photolabile Attachment And Release For Desorption And Detection Of Analytes;” Wright, 1999, Prostate Cancer and Prostatic Diseases 2: 264-76 and Merchant and Weinberger, 2000, Electrophoresis 21: 1164 67. Molecules (e.g., peptides) in a test sample can be ionized by any method known to the skilled artisan. These methods include, but are not limited to, electron ionization, chemical ionization, fast atom bombardment, field desorption, and matrix-assisted laser desorption ionization (“MALDI”), surface enhanced laser desorption ionization (“SELDI”), photon ionization, electrospray, and inductively coupled plasma.

A variety of mass spectrometry systems can be employed to analyze sample molecules captured using the methods of the invention. Mass analyzers with high mass accuracy, high sensitivity and high resolution can be used and include, but are not limited to, matrix-assisted laser desorption time-of-flight (MALDI-TOF) mass spectrometers, ESI-TOF mass spectrometers and Fourier transform ion cyclotron mass analyzers (FT-ICR-MS). Other modes of MS include an electrospray process with MS and ion trap. In ion trap MS, fragments are ionized by electrospray or MALDI and then put into an ion trap. Trapped ions can then be separately analyzed by MS upon selective release from the ion trap. Fragments can also be generated in the ion trap and analyzed. The sample molecules labeled with a mass tag using methods of the invention can be analyzed, for example, by single stage mass spectrometry with a MALDI-TOF or ESI-TOF system. Furthermore, LC-MS/MS or LC-ESI-TOF can be used. It is understood that any MS methods and any combination of MS methods can be used to analyze a sample molecule.

Finding stable MS patterns predictive of clinical outcomes requires dimensionality reduction of the MS data. The process of dimensionality reduction translates the original data into a few “composite” features which reflect most of the information of the original data. Alternatively, feature selection operates on either the original features (peptide peaks) or composite features, selecting those features which carry most of the information (variances) of the original data. There are several dimensionality reduction techniques available. Both filter based and embedded (wrapper) techniques are applicable.

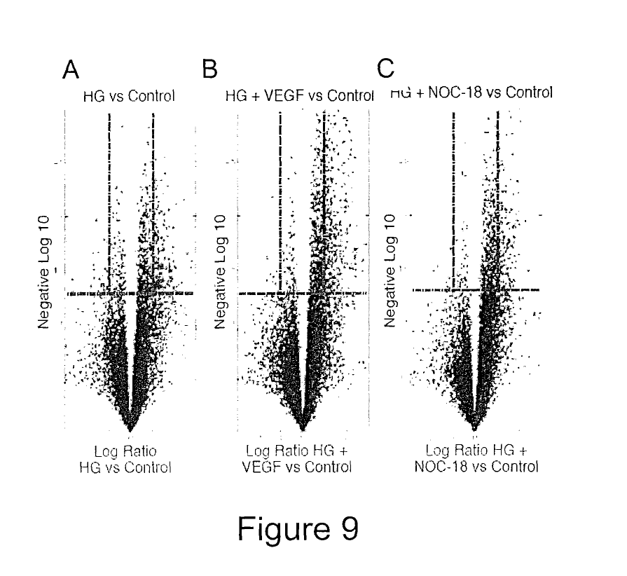

Filtering techniques encompassed by the invention that can be used to reject noise include but are not limited to temporal, spatial, and frequency domain filtering. Spatial filtering requires collecting emissions from a small area to reject noise from surrounding sources. Such confocal techniques, for instance with the target in the focus of an objective and/or using a pinhole arrangement, allow scanning of a target to reduce unwanted noise due to emissions from the material surrounding the target area. Filtering can also be achieved by applying statistical analyses to the MS peaks. One such example is applying straightforward t-tests of the MS peaks. Vulcano plots of LC-MS peptide peaks are a widely accepted graphical representation of expression data from microarray analysis that globally demonstrates differentially expressed genes. In this invention, either genes encoding the peptides of interest or peptide peaks from MS data may be used as the unit of analysis. For each peptide peak, a point is plotted based on the Log₂ of the fold change ratio for two conditions on the X-axis (e.g. hyperglycemia vs. control; hyperglycemia+nitric oxide donor vs. hyperglycemia) and the negative Log₁₀ of the P-value on the Y-axis, creating the effect of an erupting volcano from which this graphical analysis derives its name. The utility of this type of analysis is their ability to illustrate significant differences in selected differentially expressed peptides as evidenced by the asymmetry of the plots (FIG. 9).

Another useful statistical method for reducing the dimensionality of MS data is the Principal Component Analysis (PCA) and Partial Least Squares Discriminant Analysis (PLSDA) Classification (clustering) method. These methods group samples with common protein profiles based on principal component analysis, multivariate regression analysis and pair-wise comparisons of treatment groups for changes in protein analysis.

Alternatively, it is possible to embed the feature selection step within the classification process, such as filtering features based upon a “variable importance measure,” a byproduct of the Random Forest (RF) classification program. An alternative method for embedded feature selection for MS data uses a sequential forward selection (SFS) algorithm in conjunction with a nearest centroid classification algorithm.

In the present invention, without wishing to be bound by any particular theory, total ion chromatograms may be subjected to data preprocessing algorithms (e.g. MZmine) to crop data sets, filter the data through Savitzky-Golay and chromatographic medium filters and a recursive threshold-peak picker, a peak-pair aligner and a peak-normalizer to align and compare peak intensities across all samples simultaneously.

The invention also provides methods and compositions for labeling molecules in a sample by capturing the molecules on a solid support with a chemical group that allows transfer of desirable functional groups, including tags useful for enhanced detection and to facilitate identification and quantitation of tagged molecules, to the molecules. The methods are advantageous in that they can be used to selectively isolate and label molecules from a sample, allowing quantitative analysis of complex mixtures of analytes, including analysis by methods such as mass spectrometry. Thus, the methods can be used to isolate essentially all of a particular class of molecules or a subset of molecules, for example, essentially all polypeptides or the subset of phosphoproteins, glycoproteins, or otherwise modified polypeptides.

Using general covalent capture-and-release chemistries, specific functional groups can be transferred to the components of a complex sample. Furthermore, by incorporating the ability to release a captured molecule, the methods can also advantageously be used to isolate or purify sample molecules, which can be useful for reducing the complexity of a sample being analyzed. The methods are well suited for quantitative proteome analysis, for the systematic and quantitative analysis of protein phosphorylation and other post translational modifications and can be extended to the systematic and quantitative analysis of molecules other than proteins and peptides. Moreover, the methods of the invention are advantageous in that sample molecules can be efficiently captured and released, allowing the use of smaller amounts of starting sample, which is particularly useful for analyzing complex biological samples for proteomics analysis.

### Bioinformatics

The bioinformatics/biostatistics analyses assist in the search for biomarkers linked to abnormalities of cardiovascular development, as well as biomarkers indicative of a diabetic state in early pregnancy.

The current invention includes, but is not limited to, applying a statistical approach between protein level distributions between hyperglycemic/diabetic mothers and normoglycemic mothers; examining correlation between serum protein levels and amniotic fluid protein levels; correlation of 3-D yolk sac volumes with protein level expression; differential protein level expression predictive of a positive oral glucose tolerance test and cardiac developmental abnormalities. These analyses establish the behavior of the relevant biomarkers in various physiological conditions. The biostatistics-bioinformatics analysis is implemented by validating the differential expression of candidate proteins in diabetic and non-diabetic samples. The Kolmogorov-Smirnov test of normality (R function), the student t-test and/or the Mann-Whitney test are used as appropriate, to measure a significant difference of the means in any candidate protein. The student t-test also enables the calculation of the power to detect various differences of the means between hyperglycemic and normoglycemic populations. Detecting a significant difference of the means, in any of the candidate proteins, validates the protein as being differentially expressed under diabetic conditions.

ELISA and Western blot measurements of candidate proteins further validate measurements of candidate proteins in serum and amniotic fluid obtained from different populations. In order to reduce the influence of outliers, the Spearman rank order correlation using the R (short for R project for statistical computing) function is used. An r>0.8 indicates reasonable correlation. Candidate proteins are also validated using uni- and multivariate analysis for correlation with 3-D ultrasound measurements as an objective indicator of hyperglycemic insult to the embryo. Thus, the 3-D volume of the yolk sac is correlated with the protein expression panel. This problem is addressed as a linear regression problem; the association between the yolk sac volume and the candidate proteins is assessed. Regression models with 1 to n model variables, including all variable combinations will be assessed. Exhaustive searches will be made possible by porting R scripts to a parallel processing environment on the Yale Pathology linux cluster computer. Prediction error of the regression models will be assessed through a resampling procedure (Molinaro, 2005, Bioinformatics, 21:3301-3307): the samples are split into 5 equal parts, using 4 parts as the learning set, and the remaining part as the test set. The setup allows for an extra-sample estimate of the prediction error, by fitting a model on the training set, and estimating the prediction error on the test set (across 5 different folds). The straightforward loss function will be used:

L(Y,\hat{f}(X))=(Y−\hat{f}(X))²,

the squared error of the difference between the true yolk sac volume y and the predicted volume f(x). For model selection (the optimal variable combination with 1 to n variables), an additional re-sampling procedure is used, nested within the prediction error estimation step. For above tasks, the R lm function for fitting linear regression models is used. To expand the previous analysis to clinical outcome variables in the offspring, a similar re-sampling procedure as discussed is used. The dataset deals with binary outcome variables (positive OGTT and evidence of heart disorder), thus logistic regression models (R lrm function from the design library in R) are used. A typical loss functions for logistic regression models is the model misclassification rate, the ratio of incorrectly classified samples vs. all samples. The measure corresponds to 1 minus the model (or test) accuracy A

$$A = \frac{TP + TN}{TP + TN + FP + FN} .$$

The test sensitivity [TP/(TP+FN)] and specificity [TN/(TN+FP)] are also measured. Another and related model performance measure is the Receiver Operating Characteristics (ROC) analysis. Instead of using a fixed cutoff, the ROC measure evaluates a continuous range of model predictions

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

### Conceptus Isolation and Culture

Timed pregnant mice are used to harvest 7.5 and 9.5 days post conception (dpc) conceptuses from which yolk sac and hearts are dissected. The atrioventricular canals are isolated for subsequent culture and analysis of the epithelial to mesenchymal transformation that occurs in the formation of the endocardial cushion.

Conceptuses are cultured for 48 hrs in the presence of rat serum or rat serum+20 mM D-Glucose, or rat serum+20 mM D-Glucose+rVEGF or NO donor or 20 mM mannitol for 48 hrs. At 8.5 dpc the yolk sacs are harvested and analyzed using LC-MS and LC-MS-MS methods as well as by traditional cell and molecular biological methods. At 9.5 dpc atrioventricular (AV) canals are harvested from the conceptuses and cultured for 48 to 72 hrs on collagen gels to allow epithelial-mesenchymal transformation (EMT). The AV canal cultures are analyzed by double or triple label standard and confocal immunofluorescence microscopy, laser dissection capture microscopy followed by Western blotting for protein determination, characterization and localization. The effects of exogenous VEGF-A (1, 10 & 20 ng/ml), Pro- and mature BDNF (1, 25 & 50 ng/ml), NO donor (Noc-18) TIMP-1 and TIMP-2 (1, 25 & 50 ng/ml) on EMT and protein expression, post-translational modification and degradation are assessed.

### Streptozotocin-Induced Diabetes

Streptozotocin-induced diabetes in female mice is a well established model used to study the effects of hyperglycemia in pregnancy. Specifically, six to eight week old, female mice (CD1 and C57BL/6 wildtype) are injected IV with 250 mg/kg streptozotocin (Sigma Chemical Co., St. Louis, Mo.) freshly dissolved in 5 mM citrate buffer pH 4.5 IP prior to mating. Control animals receive an equivalent volume of buffered citrate solution. Streptozotocin is an antibiotic, approved by FDA for human use in certain pancreatic carcinomas, but is known to induce diabetes in rodents. Secondary to its fast elimination, no toxic effect is reported on the developing conceptuses. Animals are screened for the onset of diabetes every other day by testing urine for the presence of glucosuria. The majority of streptozotocin-treated mice become diabetic within 1-2 weeks. The diabetic animals are free of pain and no streptozotocin-related complications are expected. The animals are in good health, but they need extra daily water supply. Diabetic animals will receive daily injections of 1 to 2 units of human recombinant insulin for proper diabetic control prior to and one day following mating. Conceptuses will be harvested from control and streptozotocin-treated non-diabetic and diabetic mothers and collected at day 9.5 p.c. and subjected to morphological and biochemical analyses.

### LC-MS and MS-MS Analyses

Protein extractions from 8.5 dpc control, hyperglycemic and hyperglycemic treated yolk sacs are lysed and peptides are generated by trypsin digestion. The peptide samples are desalted using C-18 columns. LC-MS and LC-MS-MS analysis is performed on a Q-TOF API-US quadrupole time of Right tandem mass spectrometer (Waters, Milford, Mass.) equipped with a nonospray source. The instrument affords typical accurate mass measurements in the range 5-10 ppm at resolution of 10,000 that is more then adequate for a wide variety of proteomic applications. Tryptic peptides are eluted into the source following chromatography on a capillary HPLC CapLC system equipped with a splitting device affording flow regimes in the 50-300 ml/min range. For nonoflow based mode (flow set at 100 mL/min) ionization methods, a linear 30 min gradient of increasing acetonitrile concentration is applied to fractionate and elute the samples. The mass spectrometer source voltage for this application is set at 3.5 kV with scan time duration of Is/scan and spectra are acquired using both survey and parent scan MS methods with data dependent acquisition (DDA) setting defined by MassLynx software instrument interface. Following the acquisition, data are preprocessed automatically by MassLynx and MZmine (for detection and deconcolution of peak ratios) and the resulting MS/MS spectra are submitted to MZmine, SeQuest and Mascot sequence database search tools. Protein identification and post-translational modification mapping is performed by including specific mass modifications in the search criteria of the algorithms. Fragmentation spectra are searched against the nonredundant protein sequence database (NCBI, National Center for Biotechnology) and SWISSPROT protein databases. To increase confidence in protein abundance ratio standard and multivariate statistical treatment is applied to normalized ion intensities as previously described (Davidov, 2003, Drug Discov. Today, 8:175-183; Clish, 2004, OMICS, 8:3-13).

To increase confidence in protein abundance ratio standard and multivariate statistical treatment is applied to normalized ion intensities. Because raw spectrum data invariably contains “noise” from multiple nonspecific ion events post-filtering using algorithmic treatment becomes necessary. Noise subtraction and smoothing of spectra can be accomplished by applying data entropy-based peak and mass trace detection algorithm to each dataset to extract peak intensity and signal quality information. Processed in this fashion the data now yield quantifiable abundance ratios for all included mass to charge (m/z) signals that had intensities above the noise threshold that was preset in the post-acquisition processing A three-tiered classification method is performed to isolate significantly different ratios which will then be identified using SeQuest and Mascot search engines (Lee, 1995, Diabetes, 4:20-24; Ilan, 2000, J. Biol. Chem., 275:21435-21443).

### Bioinformatics/Biostatistics

The bioinformatics/biostatistics analyses assist in the search for biomarkers linked to abnormalities of cardiovascular development, as well as biomarkers, which are indicative of a diabetic state in early pregnancy. The biomarker discovery process is approached from two angles: First, culture models of cardiovascular development are used for assessing protein dysregulation under hyperglycemic conditions. Also, a diabetic animal model is used for pinpointing protein markers of diabetes during pregnancy. Data analysis focuses on a) the identification of differentially expressed proteins via MS analysis of material from both culture and animal models, and b), assessing the prognostic potential of enzyme and western blot assays of validated proteins from step a) on human amniotic fluid (AF) and serum samples, for predicting biological meaningful endpoints (such as yolk sac volume, presence of cardiac abnormalities, and positive glucose tolerance test, respectively). Finally c), human samples are also subjected to MS analysis, which enables the algorithmic discovery of prognostic MS profiles. Here, the necessary bioinformatics steps for finding differentially expressed proteins in the culture and mouse samples are presented.

Enzyme and Western Blot Assays with Candidate Proteins

Candidate proteins are validated for detecting early cardiovascular insults in the human embryo (as measured by yolk sac size and incidence of cardiac abnormalities in the anatomical ultra sound). Candidate proteins from the streptozotocin (STS)-induced diabetic animal model are assessed for their prognostic value of a positive oral glucose tolerance test (OGTT), with the rational of providing an early test for gestational diabetes. These analyses are performed on both the maternal serum and AF, if available. Below, is the statistical approach, using the prediction of 3-D yolk sac volume, positive oral glucose tolerance test (OGGT) and cardiac abnormalities as the leading examples.

Specifically examined is 1) whether the protein level distributions are different between diabetic and control patients, 2) whether the protein levels are correlated with 3-D yolk sac volumes, 3) whether they are predictive for a positive OGGT, as well as 4) cardiac abnormalities. The key is to compare different serum sample pairings (such as diabetic and pregnant patients vs. pregnant controls). Careful consideration is paid to the different populations, i.e., pregnant patients with diabetes mellitus (DM) Type 1, 2 or gestational diabetes. These analyses establish behavior of the markers under above conditions.

### Bioinformatics Strategies for Analyzing the MS Data for Biological Significance

MS data is subjected to bioinformatics analyses in order to find biological explanations for the differentially expressed proteins. Standardized ontologies (such as Gene Ontology; Ashburner, 2000, Nature Genetics, 25:25-29) and data from protein-protein interaction databases for inferring biological functions, processes and pathways of the differentially expressed proteins are all used. As shown earlier, highly significant proteins from the MS runs are mapped onto Gene Ontology classes, for grouping proteins according to various biological functions such as protein receptors or transcription factors. Statistical tests (such as those based on the hypergeometric distribution) are useful to pinpoint significant Gene Ontology classes in an automated fashion (Al-Shahrour, 2004, Bioinformatics 20:578-580). Another powerful approach for deriving biological meaning from proteomic data is the mapping of differentially expressed proteins to molecular interaction networks. The basic idea is to identify network clusters of interacting protein regions, which contain many of the differentially expressed proteins. Such clusters may represents the pathways (either known or unknown) that are behind the pathological response (such as development of cardiovascular abnormalities). The task of identifying these network clusters can be achieved by a process called Molecular Triangulation, which has been described previously (Krauthammer, 2004, Proc. Natl. Acad. Sci. USA, 101:15 148-15153). Also, overlap of those network clusters with known pathways may give additional hints for disease pathogenesis.

The results of the experiments presented in this Example are now described.

### Example 1

### High α-D Glucose Levels Arrest Yolk Sac Vasculogenesis and is Correlated with VEGF and NOS Isoform Switching

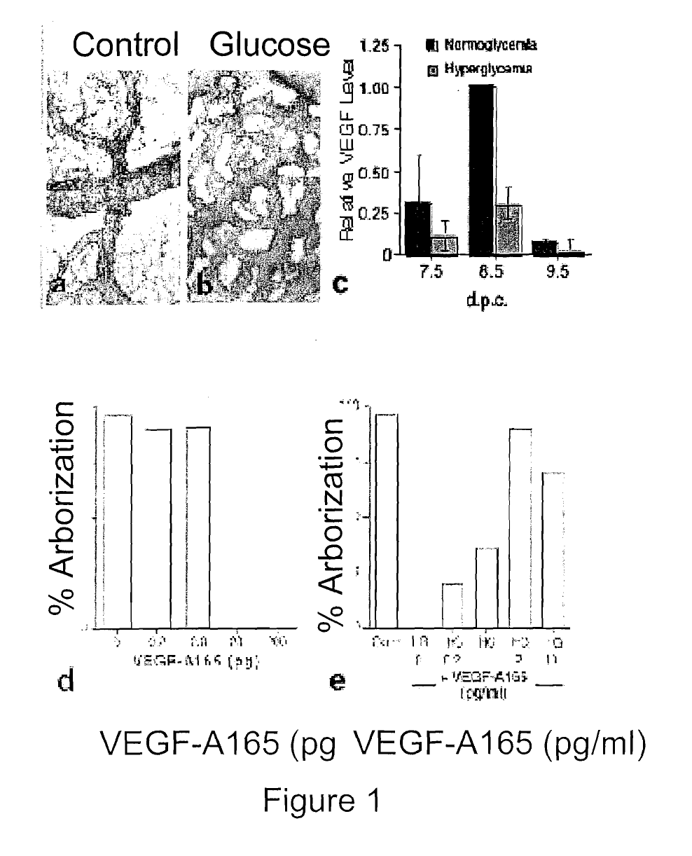

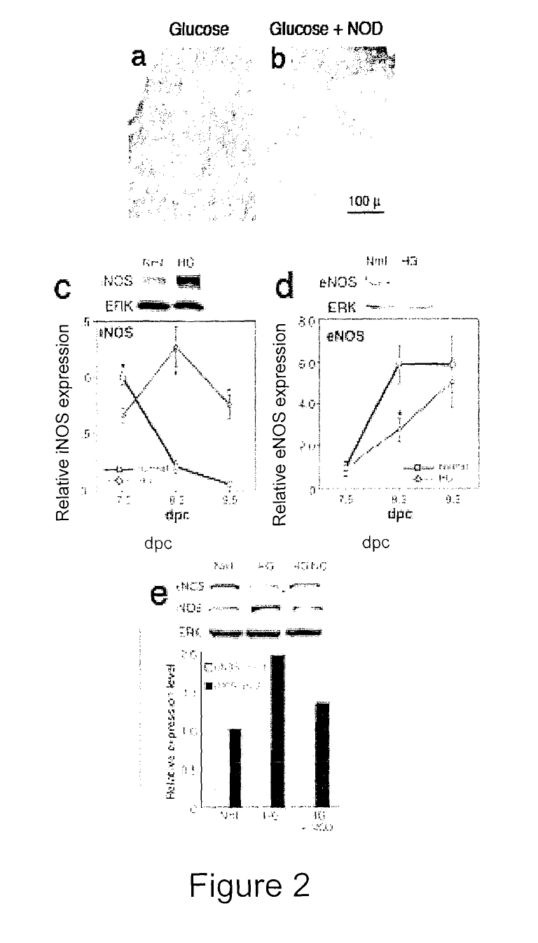

Congenital cardiac anomalies are among the most devastating defects observed in children born to diabetic mothers. These defects are largely defects that arise from disruptions of early organogenesis. In order to more rigorously study the effect of hyperglycemia on cardiac organogenesis and vasculogenesis, conceptus cultures were exposed to either normoglycemic or hyperglycemic media. Transient (as little as 3 hr) high levels of alpha-D-glucose (20 mM, similar to those measured in diabetic mothers) and NOS inhibitors arrest yolk sac vasculogenesis at the primary capillary plexus stage (FIG. 1A & FIG. 1B). Note the lack of vessel arborization in the hyperglycemia exposed conceptuses. These effects correlate with dysregulation of: VEGF expression levels from the endodermal cells (FIG. 1C); persistent tyrosine phosphorylation of PECAM-1 and SHP-2/PECAM-1 interactions in the differentiating endothelial cells (Pinter, 1999, Am. J. Pathol., 154:1367-1379; Pinter, 1997, Am. J. Pathol., 150:1523-1530; Pinter, 2001, Am. J. Pathol., 158:1199-1206); and dysregulation of eNOS/iNOS switching in endoderm and mesoderm (Nath et al., 2004, Development, 131:2485-2496) (FIG. 2A through FIG. 2D). This glucose-induced vasculopathy can be reversed by adding either exogenous VEGF-A₁₆₅ (FIG. 1D & FIG. 1E) or an NO donor (FIG. 2A through FIG. 2E), implicating both NO and VEGF as crucial interacting modulators of early vasculogenesis (Pinter, 1999, Am. J. Pathol., 154:1367-1379; Nath, 2004, Development, 131:2485-2496; Pinter, 1997, Am. J. Pathol., 150:1523-1530; Pinter, 2001, Am. J. Pathol., 158:1199-1206).

### Example 2

### Effects of Hyperglycemia and VEGF and NO Donor Intervention on Early Embryo Cultures

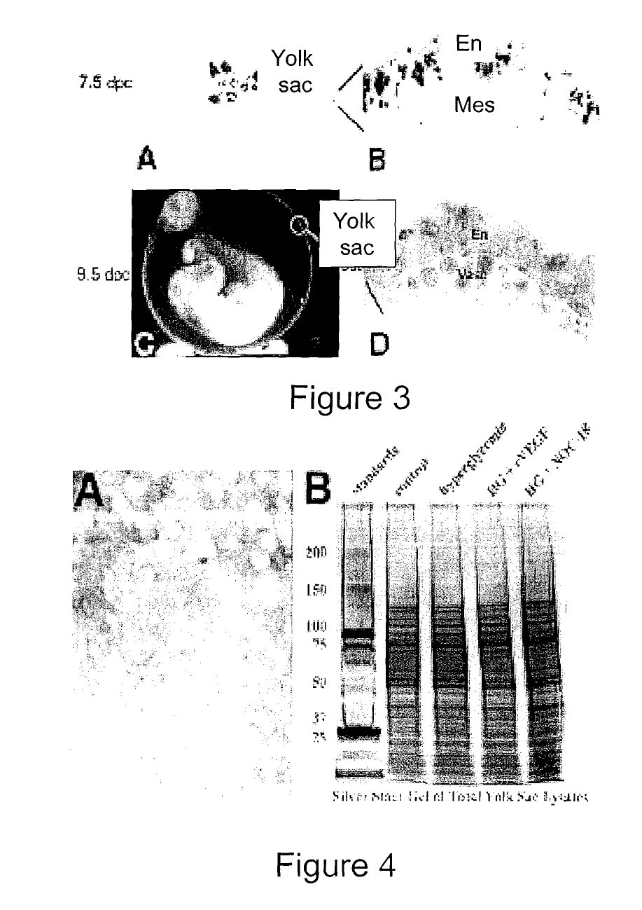

Early rodent embryo isolation, staging and culture and mass spectrometry-based proteomic analyses requires isolation and culture of murine conceptuses at 7.5 dpc (FIG. 3) and cardiac outflow tract and atrioventricular canals at 9.5 dpc (not shown) (Pinter, 1986, Teratology, 33:73-84; Pinter, 1999, Am. J. Pathol., 154: 1367-1379; Nath, 2004, Development, 131:2485-2496; Pinter, 1997, Am. J. Pathol., 150:1523-1530; Pinter, 2001, Am. J. Pathol., 158:1199-1206; Hallaq, 2004, Development, 131:5197-5209).

Using this embryo culture system, groups of ten 7.5 dpc conceptuses were cultured in normoglycemic conditions (5.0 mM D-glucose [control]), hyperglycemic conditions (20 mM D-glucose [HG]), HG+10 pg/ml VEGF A₁₆₅ (HG+rVEGF) and HG+a slow release NO donor (HG+Noc-18) for 24 hrs. The conceptuses were harvested at 8.5 dpc when their yolk sacs were at the primary capillary plexus stage and beginning to undergo vascular remodeling. Lysates were then prepared for SDS-PAGE, Silver Staining and MS analysis. FIG. 4A depicts PECAM-1 staining of the capillary plexas at 8.5 dpc and illustrates the non-descript vascular plexus observable at this stage of development. No appreciable differences in the morphology of PECAM-1 labeled capillary plexus was observed at this stage of development. Lysates run on one-dimensional SDS-PAGE electrophoresis also did not demonstrate differences between these various conditions. This is in contrast to LC-MS and LC-MS-MS analyses of these samples (see below).

### Example 3

### LC-MS and LC-MS-MS Analysis Yields Identification of Biomarkers Specific For Cardiovascular Development (Specifically Yolk Sac Vasculo- and Angio-Genesis and Cardiac Cushion Development) and their Dysregulation in the Offspring of Maternal Diabetics

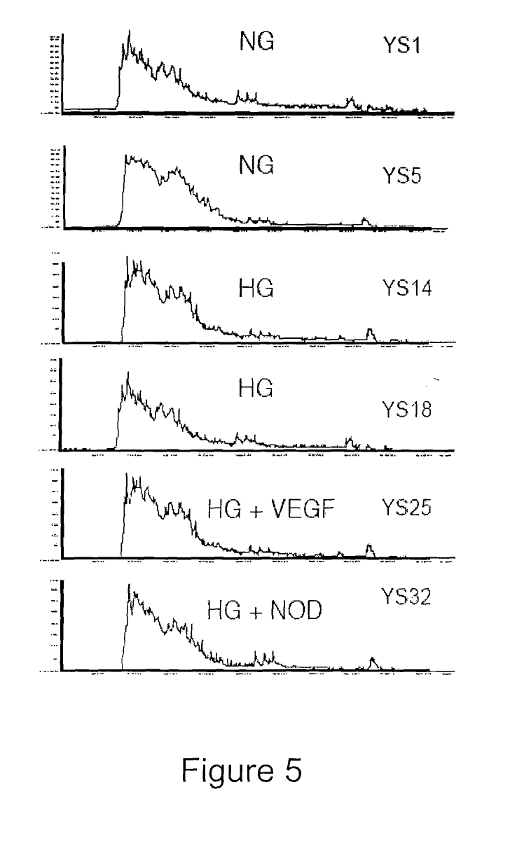

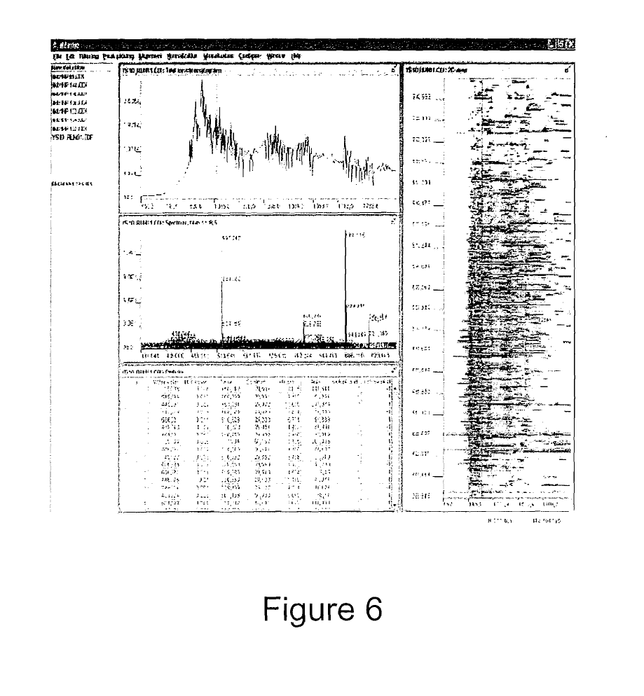

Although a great deal has been learned regarding the mechanisms involved in embryonic vasculo- and angio-genesis and cardiac cushion formation, there is a relative paucity of studies concerning the underlying mechanisms at play in these processes in the offspring of diabetic mothers. To address this, a protein profiling approach utilizing LC-MS and LC-MS-MS coupled with a cellular, molecular and biochemical approach using known antibody and nucleic acid reagents specific for proteins known to be involved in cardiovascular development was used. Specifically, sets of individual 8.5 dpc yolk sac samples and 10.5 dpc cardiac cushion cultures (euglycemic, hyperglycemic, hyperglycemic—treated with rVEGF and hyperglycemic—treated with NO donor) were lysed and subjected to multi-dimensional chromatography and mass spectrometry. The total ion chromatograms consisting of the peptide mass spectra derived from the yolk sac or cardiac cushion extracts (FIG. 5) were then subjected to data preprocessing algorithms consisting of MZmine software to crop data sets, filter the data through Savitzky-Golay and chromatographic median filters and a recursive threshold peak picker, a peak-pair aligner and a peak normalizer to align and compare peak intensities across all samples simultaneously (Davidov, 2003, Drug Discov Today, 8:175-183; Katajamaa and Oresic, 2005, BMC Bioinformatics, 18:179; Katajamaa, 2006, Bioinformatics, 22:634-636; Davidov, 2004, OMICS, 8:267-288; Oresic, 2004. Appl Bioinformatics, 3:205-217; Curtis, 2005, Trends Biotechnol, 23:429-435.). This analysis combines intensity plot, selected MS spectra view and peak tables as illustrated in FIG. 5 and FIG. 6.

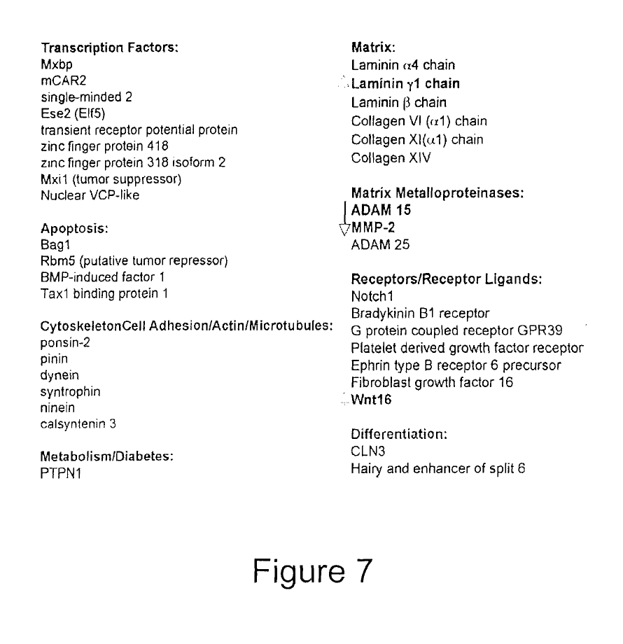

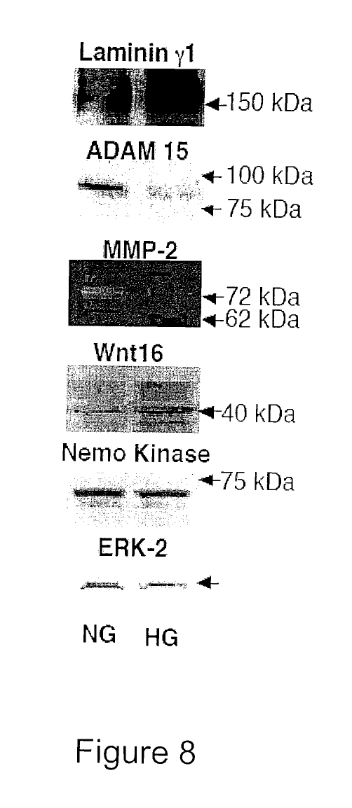

Data derived from these analyses were used for the detection of differentially expressed proteins (FIG. 7). Out of a total intensity matrix of 13,000 MS peaks in the experiment discussed above, 651 peaks showed significant changes (p<0.05, fold greater/less than +/−2). 211 of these showed differential expression in hyperglycemic vs normal samples. Using MASCOT and SEQUEST the respective proteins were identified and a subset were validated by Western blotting (FIG. 8). The identification of these peaks allows the evaluation of treatment-specific proteins as potential biomarkers and/or potential therapeutic targets.

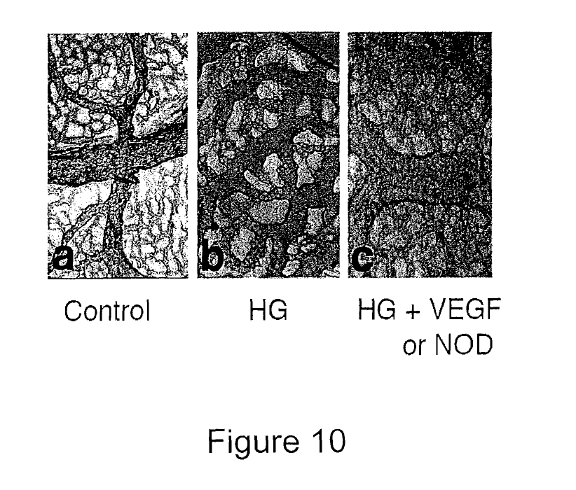

A systematic search for additional differentially expressed proteins, prioritizing protein identification according to peptide peak fold change and p-values (see Volcano plots in FIG. 9) revealed 143 significant peaks that, while dysregulated by hyperglycemic treatment, exhibited a return to euglycemic levels with treatment with either rVEGF or NO donor. These differences are illustrated in representative Vulcano plots (FIG. 9). The volcano plot is a widely accepted graphical representation of expression data from microarray analysis that globally demonstrates differentially expressed genes. In the plots depicted in FIG. 9, genes are replaced by peptide peaks as the unit of analysis. Specifically, upon analysis of the normoglycemic group (n=10) and the hyperglycemic group (n=10) differences were noted. For each peptide peak, a point is plotted based on the Log₂ of the fold change ratio for two conditions on the X-axis and the negative log₁₀ of the P value is plotted on the Y-axis. This creates the effect of an erupting volcano, from which the plot derived its name. These particular plots illustrate significant differences in selected differentially expressed peptides as evidenced by the asymmetry of the plots. Namely, in hyperglycemia vs. control samples there are 211 peaks that are either up- or down-regulated following hyperglycemic treatment. Following treatment of hyperglycemic samples with either rVEGF-A₁₆₅ or NOC-18, 143 of these peaks are returned to their normoglycemic (control) levels and morphological examination of similarly treated cultured Yolk Sacs revealed rescue of the hyperglycemic arrest of vascular development (FIG. 10).

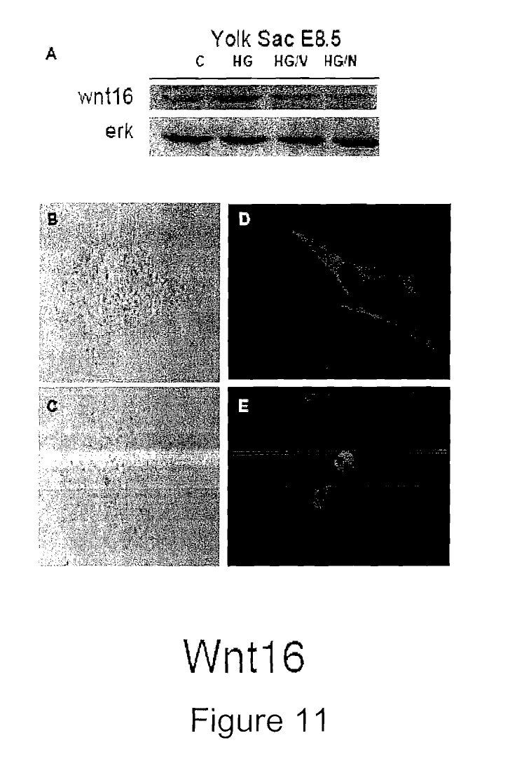

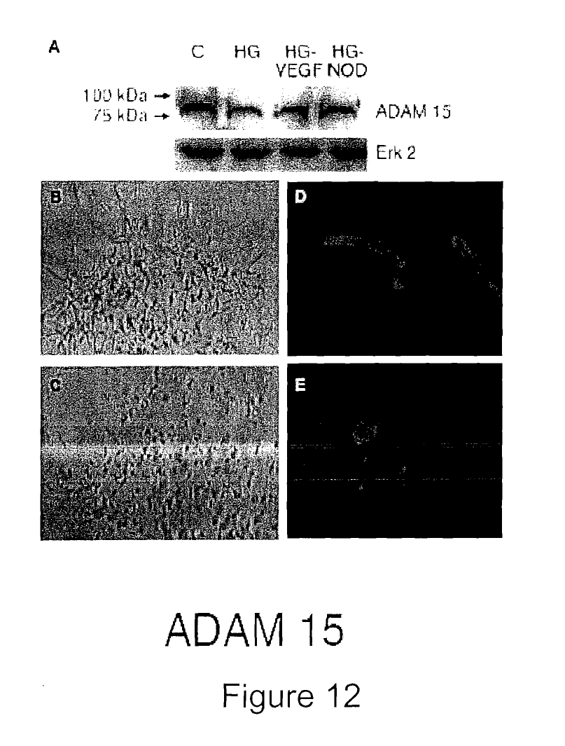

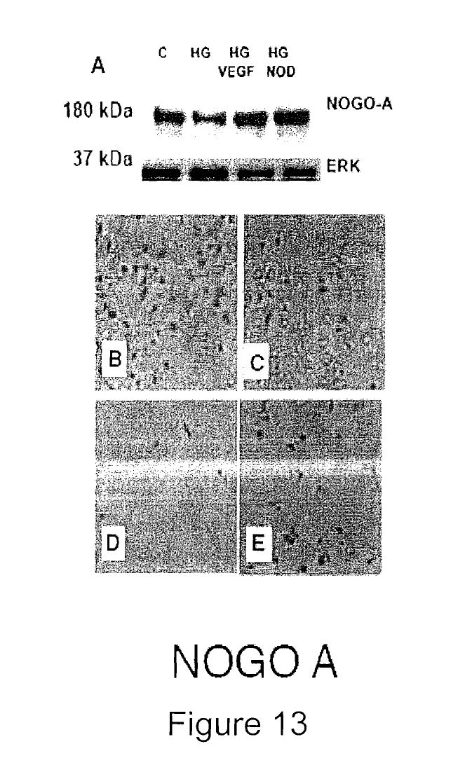

Further, several of these proteins were validated in conceptus culture assays prior to and following VEGF and NO donor rescues using Western blotting, histological and immunolocalization analyses. Specifically, expression levels of Wnt16 (FIG. 11), ADAM 15 (FIG. 12), and NOGO A (FIG. 13) were found to be rescued by addition of either VEGF or NO donor to hyperglycemic yolk sac cultures. Further, sequestration of Wnt16 (FIG. 11), ADAM 15 (FIG. 12), and NOGO A (FIG. 13) by antibodies directed against them abrogated EMT in cardiac AVC cushion explant cultures. Additionally, Western blotting, Northern blotting and in situ hybridization of yolk sacs and cardiac tissues harvested from euglycemic and STREPTOZOTOCIN (STZ) induced diabetic timed-pregnant dams and euglycemic and hyperglycemic cultures of yolk sacs and cardiac cushions were performed (Data not shown).

Potentially valuable biomarkers discovered through the LC/MS assay were further validated by replicating the initial analysis conditions by employing two alternative LC/MS platforms. Two commonly used tandem mass spectrometry platforms that predominate proteomics discovery space are QqT of (quadrupole time of flight) and QIT (quadrupole ion trap) configurations. In the recent comparison study complex protein mixtures were analyzed in parallel with QqT of and QIT instruments (Elias, 2005, Nat. Methods, 2:667-675). Despite the differences in acquisition parameters and the length of acquisition times the numbers of proteins successfully identified from each instrument were essentially the same. Thus, validation process can be vastly improved by use of complementary systems and application of more restrictive scoring criteria for biomarker discovery process.

Exploiting the fundamental differences in mass accuracy, resolution and dynamic range between these two systems generates complementary sets of data thereby gaining more confidence in the initial screen results obtained on the QqT of mass spectrometer. The addition of orthogonal analytical techniques to the validation process aided in confidently characterizing potential biomarkers for their ability to provide clinically meaningful measurements beneficial to patient outcomes.

These changes are consistent with the arrest of vasculogenesis in the yolk sac and arrest of epithelial to mesenchymal transformation in the endocardial cushion areas of the heart. The increase in Laminin γ1 chain expression following hyperglycemic insult is consistent with previous data on basement membrane induction in experimental diabetes and consistent with the observed stabilization of the primary capillary plexus and lack of vascular remodeling observed following this insult in vitro and in vivo (Nielson, 2005, Diabet. Med., 22:693-696; Eriksson, 1991, Diabetes, 40:94-98; Kitzmiller, 1996, Diabetes Care, 19:514-541; Ellington, 1997, Int. J. Dev., Biol., 41:299-306; Rudge, 2000, Gynecol. Obstet. Invest., 50:108-112; Jovanovic, 2004, Curr. Diab. Rep., 4:266-272). The decrease in ADAM 15 expression following hyperglycemic insult is also consistent with an abrogation of vascular remodeling as ADAM 15 has known to be induced during periods of angiogenesis and its reduced expression following this insult (FIG. 8) correlates well with failure of remodeling of the primary capillary plexus at this stage of development, as does the reduction in MMP-2 expression and activity. Interestingly, Wnt16, a Wnt gene known to be involved in synovial joint formation via modulation of the Wnt/β-catenin signaling pathway (Guo, 2004, Genes Dev., 18:2404-2417), was observed to be up-regulated following hyperglycemic insult (FIG. 8 and FIG. 11). Since dysregulation of the Wnt/β-catenin signaling pathway is known to affect mesenchymal differentiation (Person, 2005, Dev. Biol., 278:35-48), it's possible that hyperglycemic dysregulation of this pathway in vasculo-, angio-genesis and endocardial cushion formation would have significant effects on normal development of the cardiovascular system. Nemo kinase does not appear to be modulated by hyperglycemic insult, illustrating the specificity of the response to this insult (FIG. 8). All five protein levels were normalized to ERK-2 (FIG. 8).

Interestingly, in whole conceptus cultures in the presence of 20 mM D-glucose, ADAM 15 expression is up-regulated to near normoglycemic levels upon treatment with either of the two agents known to rescue vasculogenesis: VEGF or the NO donor NOC-18 (data not shown). In contrast, Wnt16 is down-regulated to near normoglycemic levels upon treatment with either of these two agents (FIG. 11).

As described, vasculogenesis of the yolk sac is arrested when cultures are exposed to hyperglycemia (FIG. 10B). Administering either VEGF or NOD restores vascular arborization within the capillary bed (FIG. 10C).

This suggests that ADAM 15 and Wnt16 may be functionally important during this process and represent two of a growing list of proteins that have the potential of being useful biomarkers for early diagnosis and potential therapeutic targets.

Additionally, using atrioventricular canal cardiac cushion cultures demonstrated that anti-Wnt16 blocks the invasion of endocardial cells into the underlying collagen gel, confirming its role in inducing EMT. In addition, the effects of increased Wnt16 concentrations on EMT in cushion culture model are evaluated.

Further, analysis of 8.5, 9.5 & 10.5 dpc sera and 10.5 dpc amniotic fluid collected from euglycemic and STZ diabetic dams revealed decreased ADAM 15 expression in the amniotic fluid and sera of the diabetic dams, providing preliminary in vivo validation, albeit in a murine system.

Similar studies use cardiac A-V canal and outflow tract cultures to investigate the effects of transient hyperglycemia (D-glucose) on epithelial to mesenchymal transformation (EMT), a process required in the development of the cardiac cushions, which are the precursors of the cardiac valves and membranous septae (both of which are known to exhibit an increased incidence of maldevelopment in the offspring of diabetic mothers). Similar to yolk sac vasculogenesis studies, hyperglycemic insult (20 mM D-glucose) arrested cushion formation, which correlated with a dramatic reduction of VEGF expression by the underlying myocardium and a loss of MMP-2 and ADAM 15 induction. This arrest in cushion formation could be rescued by addition of exogenous VEGF (Enciso, 2003, J. Cell Biol., 160:605-615)

These data suggest that hyperglycemic insult during embryonic development elicits similar derangements in selected proteins during vascular development/remodeling/differentiation in a variety of tissues and organs, regardless of the time period that it occurs. Proteins that exhibit concurrent changes in both whole conceptus cultures (yolk sac vascular development) and cardiac outflow tract cultures (heart vascular development/remodeling/differentiation) are therefore ideal biomarkers for early to late human cardiovascular development.

### Example 4

### Extending Protomic Profiling to Murine Amniotic Fluid and Sera Samples

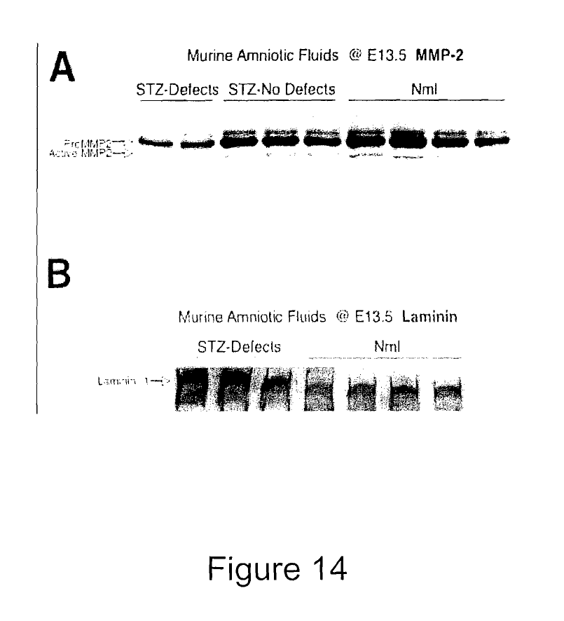

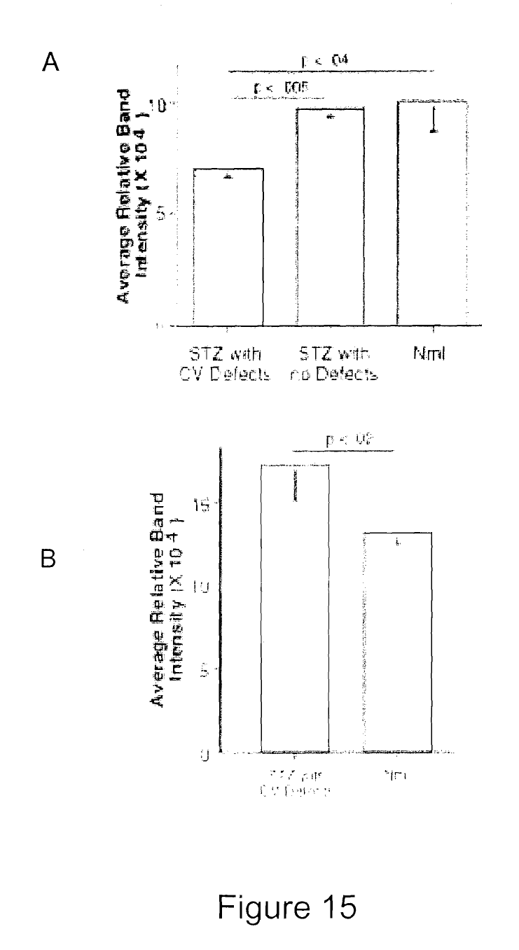

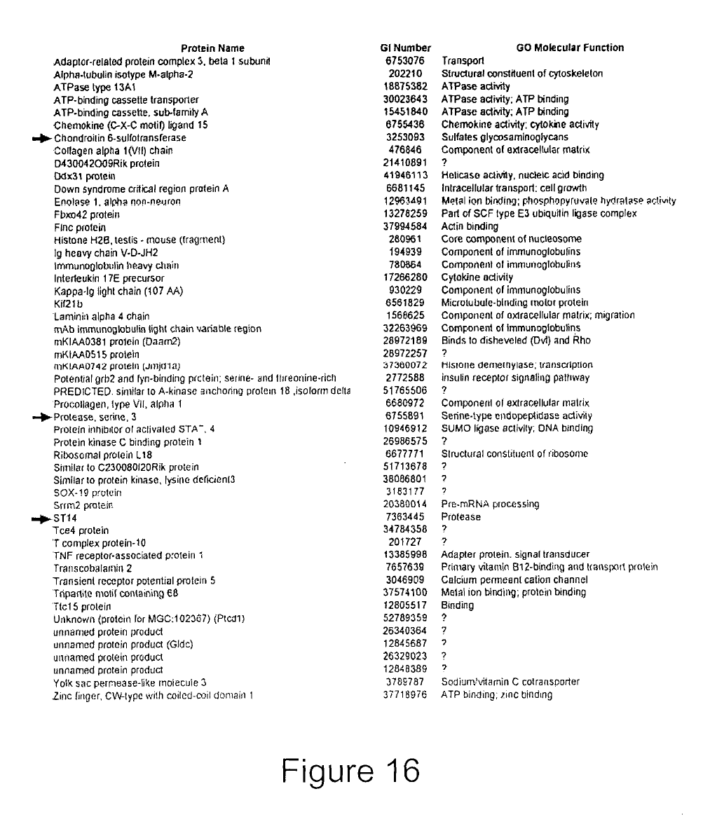

In order to validate the usefulness of the growing list of differentially-expressed proteins as biomarkers for evaluating pre- and gestational diabetes and/or congenital cardiac defects in “at risk” pregnant female murine populations, Western blot analyses were performed on E13.5 amniotic fluid samples harvested from normal, streptozoticin-induced diabetic pregnant females and streptozoticin-induced diabetic pregnant female mice carrying fetuses with cardiac defects. It was determined that while MMP-2 expression levels in E13.5 amniotic fluid samples were similar in normal and streptozotocin induced diabetic females carrying fetuses without defects, streptozotocin induced diabetic females carrying fetuses with defects exhibited decreased expression levels of MMP-2 (FIG. 14A and FIG. 15A). Additionally, Laminin γ1 chain amniotic fluid levels were found to be increased compared to those found in normal E13.5 pregnant mice (FIG. 14B and FIG. 15B).

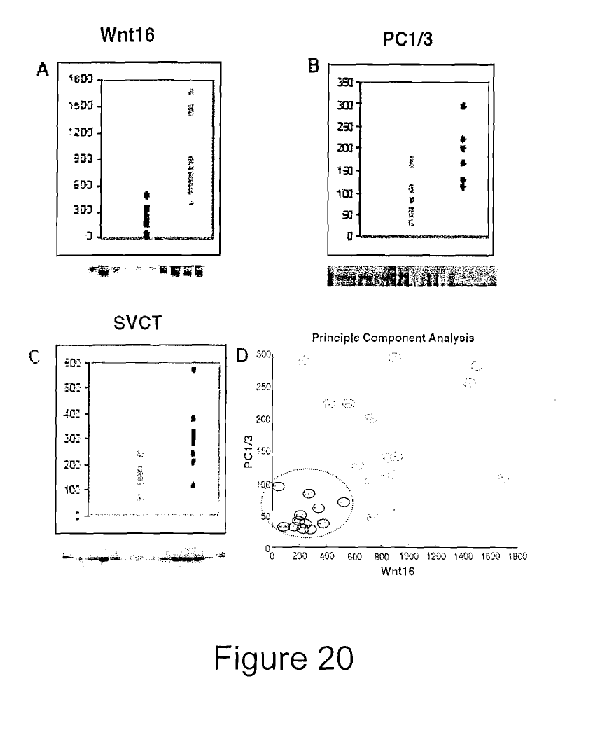

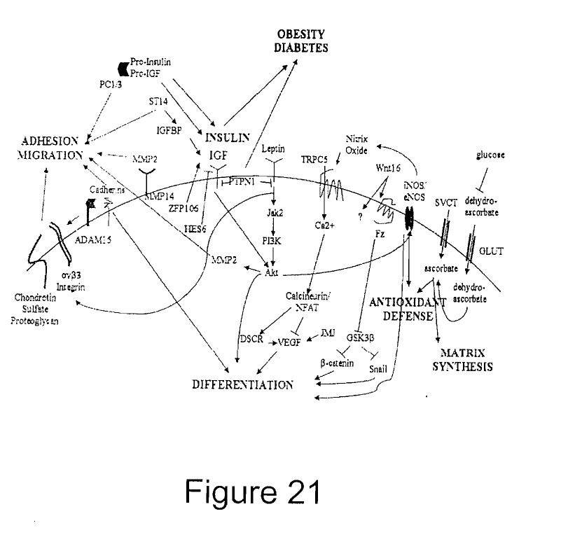

A second analysis of the dysregulated protein revealed the identification of additional proteins associated with statistically significant peptide peaks. The 143 statistically significant peptide peaks that were dysregulated by hyperglycemia but returned to normal levels by both NO and VEGF, were targeted for MS/MS fragmentation in a second round of mass spectrometry. The subsequent spectra were compared against the NCBInr database using the MASCOT search engine to identify the proteins. Proteins were then mapped to gene ontology molecular function class. Of those listed the proteins marked by heavy arrows (Chondroitin 6-sulfotransferase, Protease, serine 3 and ST14) have been found to be dysregulated in the amniotic fluid samples of 20 week gestation pregnant women carrying fetuses having known cardiac defects (see FIG. 20).

### Example 5

### Extending Proteomic Profiling to Humans

Analysis of amniotic fluid and sera has been extended to eighteen to twenty week gestation to third trimester gestation diabetic and normoglycemic women. This time period roughly correlates with the gestational time periods studied in the mouse (de Lange, 2004, Circ. Res., 95:645-654; Aikawa, 2006, Circulation, 113:1344-1352; Person, Int. Rev. Cytol. 243).

Investigators have documented continued valvular remodeling during the later stages of gestation in both the mouse and the human (de Lange, 2004, Circ. Res., 95:645-654; Aikawa, 2006, Circulation, 113:1344-1352). Hearts examined in the course of fetal and perinatal autopsies reveal that significant valvular remodeling occurs throughout gestation and is likely at risk to develop abnormally following hyperglycemic insult at virtually any time during gestation T his raises the possibility of continued risk of developing cardiovascular congenital anomalies in response to poorly controlled glucose levels throughout pregnancy (Jovanovic and Nakai, 2006, Endocrinol. Metab. Clin. North Am., 35:79-97; Jovanovic, 2005, Diabetes Care, 28:11133-1117; Loffredo, 2001, Teratology, 64:98-106; Rudge, 2000, Gynecol. Obstet. Invest., 50:108-112; Jovanovic, 2004, Curr. Diab. Rep., 4:226-272; Natrajan, 1997, A. J. Physiol., 273:H2224-H2231; Omori and Jovanovic, 2005, Diabetes Care, 28:2592-2593).

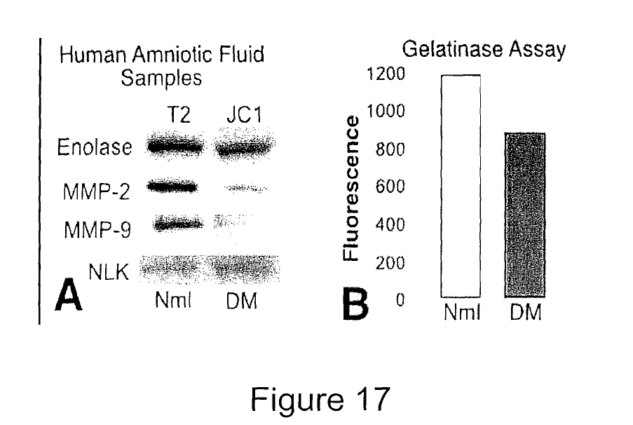

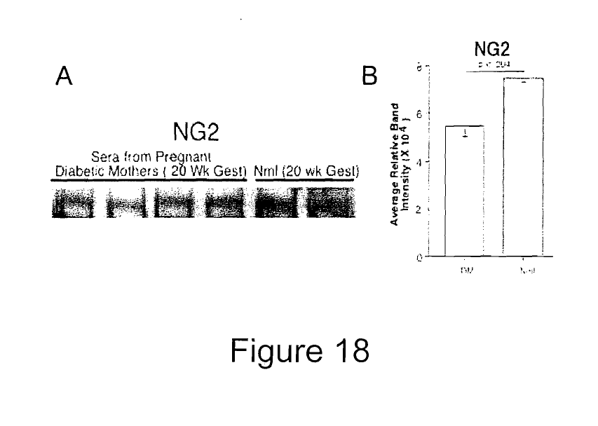

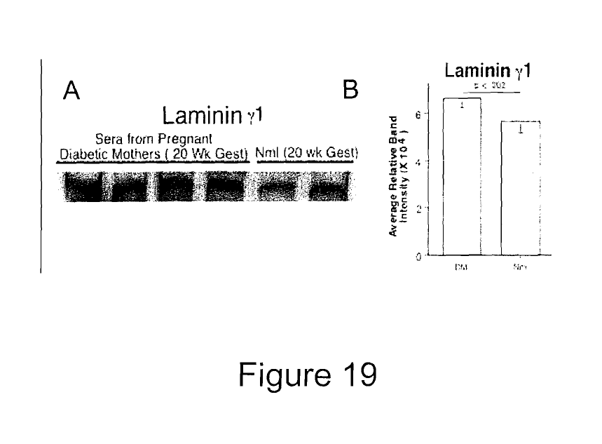

Using Western blotting to analyze a small series of amniotic fluid samples obtained from 20 week gestation normal and diabetic pregnant women, expression levels of enolase, MMP-2 and MMP-9 were decreased in the samples of diabetic women (FIG. 17). Additionally, expression of NG2 (a chrondroitin sulfate proteoglycan) was found to be decreased in diabetic samples (FIG. 18) while laminin γ1 chain expression levels were found to be increased in diabetic samples (FIG. 19).

In addition to the above-mentioned and documented protein biomarkers, using Western blotting in an ongoing larger human amniotic fluid (AF) series (twelve normal 20 week gestation AF samples and thirteen 20 week gestation AF samples with known fetal cardiovascular defects to date), increased expression levels of Wnt16 and decreased expression levels of Serine protease 3 (PC/1/3) and Sodium/vitamin C cotransporter (SVCT) were also found (see FIG. 12, A-C). Analysis of the normal AF group (n=12) and the AF group with known fetal congenital cardiac defects (CHD) (n=13), plotting PC/1/3 and Wnt16 indicates a separation of the CHD samples from the normal samples, with the normal samples forming a tight cluster (FIG. 20). Further, analysis done on this dataset using Principle Component Analysis (PCA) and Partial Least Squares Discriminant Analysis (PLSDA) Classification (clustering) methods groups samples with common protein profiles. This analysis involves principle component analysis, multivariate regression analysis and pairwise comparisons of treatment groups for changes in protein expression. It allows for multicollineality and more variables than observations.

### Example 6

### Correlation of Proteomic Profiling with Ultrasound Analyses

Ultrasound analyses of a cohort of diabetic and normoglycemic pregnant women compiled from the Sardinian population (an area known for its high incidence of diabetes in the general population) revealed differences in the yolk sac volumes and kinetics of volume change of the diabetic patients compared to that of normoglycemic mothers at 5 to 12 weeks gestation, the time period during which organogenesis occurs and the developing embryo is highly susceptible to teratogens (Cosmi, 2005, J. Perinat. Med., 33:132-136). From 5 to 10 weeks gestational age, the diabetic population exhibited increases in yolk volume compared to the control population. Interestingly, a more profound involution of the yolk sac volume was noted in the diabetic population beginning at 10 weeks gestation.

While becoming increasingly valuable as a diagnostic tool in assessing problems in the first trimester of gestation (Kupesic and Kurjak, 2001, Early Pregnancy, 5:40-41; Kupesic, 2002, J. Perinat. Med., 30:84-98; Lazarus, 2003, Radiol. Clin. North Am., 41:663-679; Kurjal, 2002, J. Soc. Gynecol. Invest., 9:186-202), the changes in yolk sac appearance are likely a result of abnormal embryonic development rather than being a cause of congenital abnormalities (Kupesic and Kurjak, 2001, Early Pregnancy, 5:40-41). Correlation of these ultrasound changes with changes in the growing list of biomarkers obtained from paired amniotic fluid and sera samples of these two populations will provide a better prognostic tool.

### Example 7

### Development of a Protein-Based Analysis to Identify at-Risk Pregnant Women Early in their Pregnancy to Reduce/Prevent the Development of Congenital Abnormalities in their Offspring

Using the data collected from protein profiling and more traditional in vivo and in vitro cell biological studies, a diverse group of proteins has been identified that exhibit robust changes in response to transient hyperglycemic insult. These include, but are not limited to: ADAM 15, MMP-2, Laminin γ1 chain, e-NOS, i-NOS, Wnt16 and leptin, enolase 1, Down's syndrome critical region protein, ST14, NG2, PC/1/3, SVCT and CH3T. Using antibodies directed against these proteins, their levels are determined by ELISA and Western blotting in amniotic fluid and/or serum samples from 7 to 12 week gestational age through the second and third trimesters.

Studies of a series of human amniotic fluid samples (total n=7) have revealed differences in the expression levels of pro-MMP-2 when comparing amniotic fluids obtained from normoglycemic patients (n=3) and controlled diabetic patients (n=2) compared to those of uncontrolled diabetics (n=2). Specifically, the amniotic fluid samples obtained from uncontrolled diabetic patients exhibited significant changes in pro-MMP-2 expression compared to samples from the other two groups as determined by zymography (Figure FIG. 17). A larger series of human amniotic fluid samples from 20 week gestation women having normal fetuses (n=12) and women with fetuses having known congenital cardiac defects (n=13) revealed significant changes in Wnt16, PC/1/3 and SVCT expression compared to samples from normal samples zymography (FIG. 20)

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of identifying a pregnant female whose fetus is at risk of developing a congenital abnormality, said method comprising measuring in a body sample obtained from said female the level of at least one biomarker, wherein when the level of said biomarker in said sample indicates that said biomarker is dysregulated in said female, said fetus is at risk for developing said congenital abnormality.

2. The method of claim 1, wherein said pregnant female is a mammal selected from the group consisting of a mouse, a rat, a non-human primate, and a human.

3. The method of claim 2, wherein said mammal is a human.

4. The method of claim 1, wherein the method comprises measuring the level of two or more biomarkers in said body sample.

5. The method of claim 1, wherein said biomarker is selected from the group consisting of a matrix metalloproteinase, a receptor, a ligand, a transcription factor, a protein affecting apoptosis, a cytoskeletal protein, a cell adhesion molecule, actin, a mictotubule protein, an enzyme, a metabolite associated with glucose metabolism, and a metabolite associated with diabetes.

6. The method of claim 5, wherein said biomarker is selected from the group consisting of laminin γ1 chain, laminin α4 chain, ADAM 15; MMP-2, MMP-9, Wnt16, enolase I t, Down syndrome critical region protein, ST14, CH3T, SVCT, NG2, NOGO A, and PC1/3.

7. The method of claim 1, wherein said body sample is selected from the group consisting of a tissue, a cell and a bodily fluid.

8. The method of claim 7, wherein said bodily fluid comprises maternal serum or amniotic fluid.

9. The method of claim 1, wherein said measuring of said biomarker comprises an immunoassay for assessing the level of said biomarker in said sample.

10. The method of claim 9, wherein said immunoassay is selected from the group consisting of Western blot, ELISA, immunopercipitation, immunohistochemistry, immunofluorescence, radioimmunoassay, dot blotting, and FACS.

11. The method of claim 1, wherein said measuring of said biomarker comprises a nucleic acid assay for assessing the level of a nucleic acid encoding said biomarker in said sample.

12. The method of claim 11, wherein said nucleic assay is selected from the group consisting of a Northern blot, Southern blot, in situ hybridization, a PCR assay, an RT-PCR assay, a probe array, a gene chip, and a microarray.

13. A method of identifying a pregnant female whose is at risk of developing hyperglycemia during pregnancy, said method comprising measuring in a body sample obtained from said female the level of at least one biomarker, wherein when the level of said biomarker in said sample indicates that said biomarker is dysregulated in said female; said female is at risk for developing hyperglycemia.

14. The method of claim 13, wherein said pregnant female is a mammal selected from the group consisting of a mouse, a rat, a non-human primate, and a human.

15. The method of claim 14, wherein said mammal is a human.

16. The method of claim 13, wherein the method comprises measuring the level of two or more biomarkers in said body sample.

17. The method of claim 13, wherein said biomarker is selected from the group consisting of a matrix metalloproteinase, a receptor, a ligand, a transcription factor, a protein affecting apoptosis, a cytoskeletal protein, a cell adhesion molecule, actin, a mictotubule protein, an enzyme, a metabolite associated with glucose metabolism, and a metabolite associated with diabetes.

18. The method of claim 17, wherein said biomarker is selected from the group consisting of laminin γ1 chain, laminin α4 chain, ADAM 15; MMP-2, MMP-9, Wnt16, enolase 1α, Down syndrome critical region protein, ST14, CH3T, SVCT, NG2, NOGO A, and PC/1/3.

19. The method of claim 13, wherein said body sample is selected from the group consisting of a tissue, a cell and a bodily fluid.

20. The method of claim 19, wherein said bodily fluid comprises maternal serum or amniotic fluid.

21. The method of claim 13, wherein said measuring of said biomarker comprises an immunoassay for assessing the level of said biomarker in said sample.

22. The method of claim 13, wherein said measuring of said biomarker comprises a nucleic acid assay for assessing the level of a nucleic acid encoding said biomarker in said sample.

23. A method of identifying an individual whose is at risk of developing hyperglycemia, said method comprising measuring in a body sample obtained from said individual the level of at least one biomarker, wherein when the level of said biomarker in said sample indicates that said biomarker is dysregulated in said individual, said individual is at risk for developing hyperglycemia.

24. The method of claim 23, wherein said individual is a mammal selected from the group consisting of a mouse, a rat, a non-human primate, and a human.

25. The method of claim 24, wherein said mammal is a human.

26. The method of claim 23, wherein the method comprises measuring the level of two or more biomarkers in said body sample.

27. The method of claim 23, wherein said biomarker is selected from the group consisting of a matrix metalloproteinase, a receptor, a ligand, a transcription factor, a protein affecting apoptosis, a cytoskeletal protein, a cell adhesion molecule, actin, a mictotubule protein, an enzyme, a metabolite associated with glucose metabolism, and a metabolite associated with diabetes.

28. The method of claim 27, wherein said biomarker is selected from the group consisting of laminin γ1 chain, laminin α4 chain, ADAM 15; MMP-2, MMP-9, Wnt16, enolase 1α, Down syndrome critical region protein, ST14, CH3T, SVCT, NG2, NOGO A, and PC1/3.

29. The method of claim 23, wherein said body sample is selected from the group consisting of a tissue, a cell, and a bodily fluid.

30. The method of claim 23, wherein said measuring of said biomarker comprises an immunoassay for assessing the level of said biomarker in said sample.

31. The method of claim 23, wherein said measuring of said biomarker comprises a nucleic acid assay for assessing the level of a nucleic acid encoding said biomarker in said sample.

32. A composition comprising a plurality of oligonucleotides attached to a substrate surface, wherein each of said oligonucleotides is a nucleic acid encoding a biomarker or a fragment thereof, or is complementary to said biomarker or said fragment thereof, wherein said biomarker is selected from the group consisting of a matrix metalloproteinase, a receptor, a ligand, a transcription factor, a protein affecting apoptosis, a cytoskeletal protein, a cell adhesion molecule, actin, and a mictotubule protein, an enzyme, a metabolite associated with glucose metabolism, and a metabolite associated with diabetes.

33. The composition of claim 32, wherein the substrate surface is a membrane, a chip, a bead, a microsphere or a microchip.

34. The composition of claim 32, wherein said biomarker is selected from the group consisting of laminin γ1 chain, laminin α4 chain, ADAM 15; MMP-2, MMP-9, Wnt16, enolase 1α, Down syndrome critical region protein, ST14, CH3T, SVCT, NG2, NOGO A, and PC1/3.

35. A composition comprising a plurality of peptides attached to a substrate surface, wherein each of said peptides is a biomarker or a fragment thereof, wherein said biomarker is selected from the group consisting of a matrix metalloproteinase, a receptor, a ligand, a transcription factor, a protein affecting apoptosis, a cytoskeletal protein, a cell adhesion molecule, actin, a mictotubule protein, an enzyme, a metabolite associated with glucose metabolism, and a metabolite associated with diabetes.

36. The composition of claim 35, where the substrate surface is a membrane, a chip, a bead, a microsphere or a microchip.

37. The composition of claim 35, wherein each of said peptides is a biomarker or a fragment thereof, wherein said biomarker is selected from the group consisting of laminin γ1 chain, laminin α4 chain, ADAM 15; MMP-2, MMP-9, Wnt16, enolase 1α, Down syndrome critical region protein, ST14, CH3T, SVCT, NG2, NOGO A, and PC1/3.

38. A composition comprising a plurality of antibodies attached to a substrate surface wherein said antibody specifically binds a biomarker or a fragment thereof, wherein said biomarker is selected from the group consisting of a matrix metalloproteinase, a receptor, a ligand, a transcription factor, a protein affecting apoptosis, a cytoskeletal protein, a cell adhesion molecule, actin, a mictotubule protein, an enzyme, a metabolite associated with glucose metabolism, and a metabolite associated with diabetes.

39. The composition of claim 38, where the substrate surface is a plate, a membrane, a solid support, a chip, a bead, a microsphere or a microchip.

40. The composition of claim 38, wherein said antibody specifically binds a biomarker or a fragment thereof, wherein said biomarker is selected from the group consisting of laminin γ1 chain, laminin α4 chain, ADAM 15; MMP-2, MMP-9, Wnt16, enolase 1α, Down syndrome critical region protein, ST14, CH3T, SVCT, NG2, NOGO A, and PC1/3.

41. The composition of 38, wherein at least one of said antibodies is attached to said substrate surface.

42. The composition of claim 41, where two or more of said antibodies are attached to said substrate surface.

43. The antibody of claim 38, wherein said antibody comprises a detectable label.

44. The antibody of claim 43, wherein said detectable label is selected from the group consisting of a radioactive, a fluorescent, a biological, and an enzymatic label.

45. A kit comprising a composition for detecting the level of a biomarker in a body sample obtained from a mammal, wherein when the level of said biomarker in said sample indicates that said biomarker is dysregulated in said individual, said individual is at risk of developing hyperglycemia, and wherein said composition comprises at least one antibody that specifically binds said biomarker or a fragment thereof, said kit further comprising instructional material for the use thereof.

46. The kit of claim 45, wherein said mammal is a human.

47. The kit of claim 46, wherein said human is a female.

48. The kit of claim 47, wherein said female is pregnant.

49. The kit of claim 45, wherein said composition comprises at least one antibody that specifically binds a biomarker or a fragment thereof, wherein said biomarker is selected from the group consisting of laminin γ1 chain, laminin α4 chain, ADAM 15; MMP-2, MMP-9, Wnt16, enolase 1α, Down syndrome critical region protein, ST14, CH3T, SVCT, NG2, NOGO A, and PC1/3.

50. The kit of claim 45, wherein at least one of said antibodies is bound to a substrate surface.

51. The kit of claim 50, wherein two or more of said antibodies are bound to said substrate surface.

52. The kit of claim 45, wherein said antibody comprises a detectable label.

53. The kit of claim 52, wherein said detectable label is selected from the group consisting of a radioactive, a fluorescent, a biological, and an enzymatic label.

54. A kit comprising a composition for detecting the level of a biomarker in a body sample obtained from a mammal, wherein when the level of said biomarker in said sample indicates that said biomarker is dysregulated in said individual, said individual is at risk of developing hyperglycemia, and wherein the composition comprises at least one nucleic acid, wherein said nucleic acid encodes said biomarker or a fragment thereof, or is complementary to said biomarker or a fragment thereof, said kit further comprising an instructional material for the use thereof.

55. The kit of claim 54, wherein said mammal is a human.

56. The kit of claim 55, wherein said human is a female.

57. The kit of claim if 56, wherein said female is pregnant.

58. The kit of claim 54, wherein said biomarker is selected from the group consisting of laminin γ1 chain, laminin α4 chain, ADAM 15; MMP-2, MMP-9, Wnt16, enolase 1α, Down syndrome critical region protein, ST14, CH3T, SVCT, NG2, NOGO A, and PC1/3.

59. The kit of claim 54, wherein said nucleic acid probe is immobilized on a solid support.

60. The kit of claim 59, wherein said nucleic acid probe is linked to a detectable label.

61. The kit of claim 60, wherein said label is selected from a radioactive, a fluorescent, a biological and an enzymatic label.
